SEQUENCE LISTING (42 sequences)

SEQ ID NO: 1 - protein, 152 aa, Artificial Sequence
  Description of Artificial Sequence: Synthetic polypeptide
ASDEEIQDVSGTWYLKAMTVDTQMPLSLYVSVTPMTLTTLEGGNLEAKVTMNQIGRSQEVKAVLEKTDEPGKYTAYGGAHVAYIIRSHVKDHYIFYSEGDTWGGPVPGVWLVGRDPKNNLEALEDFEKAAGARGLSTESILIPRQSETSSPG

SEQ ID NO: 2 - DNA, 24 bp, Artificial Sequence
  Description of Artificial Sequence: Synthetic primer
tatctgaaggccatgacggtggac

SEQ ID NO: 3 - DNA, 24 bp, Artificial Sequence
  Description of Artificial Sequence: Synthetic primer
tgcccacgagccacacccctggga

SEQ ID NO: 4 - protein, 111 aa, Artificial Sequence
  Description of Artificial Sequence: Synthetic polypeptide
ASDEEIQDVSGTWYLKAMTVDTQMPLSLYVSVTPITLTTLEGGSLEATVTLNQIGRSQEVRAVLEKTDEPGKYTAYGGAHVAYISRSHVKDHYIFYSEGDTWGGPVPGVWL

SEQ ID NO: 5 - protein, 87 aa, Artificial Sequence
  Description of Artificial Sequence: Synthetic polypeptide
VDTQTPLSLYVSVTPITLTTLEGGNLEAEVTLNQVGRSQEVRAVLEKTDEPGKYTAYGGAHVAYIIRSHVKDHYIFYSEGDTWGGPV

SEQ ID NO: 6 - protein, 87 aa, Artificial Sequence
  Description of Artificial Sequence: Synthetic polypeptide
VDPQMPLSLYVSVTPMTLTTLEGGNLEAEVTLNQIGRSQEVRAVLEKTDEPGKYTAYGGAHVAYITRSHVKDHYIFYSEGDTWGGPV

SEQ ID NO: 7 - protein, 87 aa, Artificial Sequence
  Description of Artificial Sequence: Synthetic polypeptide
VDTQMPLSLYVSVTPMTLTTLEGGNLEAEVTLNQIGRSQEVKAVLEKTDEPGRYTAYGGAHVAYIIRSHVKDHYIFYSEGDTWGGPV

SEQ ID NO: 8 - protein, 87 aa, Artificial Sequence
  Description of Artificial Sequence: Synthetic polypeptide
VDTQMPLSLYVSVTPITLTALEGGNLEAEVTLNQVGRSQEVKAVLEKTDEPGKYTAYGGAHVAYIIRSHVKDHYIFYSEGDTWGGPV

SEQ ID NO: 9 - protein, 87 aa, Artificial Sequence
  Description of Artificial Sequence: Synthetic polypeptide
VDTQVPLSLYVSVTPITLTTLEGGNLEAEVTMNQIGRSQEVRAVLEKTDEPGKYTAYGGAHVAYIIRSHVKDHYIFYSEGDTWGGPV

SEQ ID NO: 10 - DNA, 40 bp, Artificial Sequence
  Description of Artificial Sequence: Synthetic primer
cgtctcggtaacacccatatgcctcacgaccctggaaggg

SEQ ID NO: 11 - DNA, 40 bp, Artificial Sequence
  Description of Artificial Sequence: Synthetic primer
cccttccagggtcgtgaggcatatgggtgttaccgagacg

SEQ ID NO: 12 - DNA, 42 bp, Artificial Sequence
  Description of Artificial Sequence: Synthetic primer
caggtcgcacgtgaagtgccactacatcttttactctgaggg

SEQ ID NO: 13 - DNA, 42 bp, Artificial Sequence
  Description of Artificial Sequence: Synthetic primer
ccctcagagtaaaagatgtagtggcacttcacgtgcgacctg

SEQ ID NO: 14 - DNA, 37 bp, Artificial Sequence
  Description of Artificial Sequence: Synthetic primer
cgtggcatacatcagctgctcgcacgtgaaggatcac

SEQ ID NO: 15 - DNA, 37 bp, Artificial Sequence
  Description of Artificial Sequence: Synthetic primer
gtgatccttcacgtgcgagcagctgatgtatgccacg

SEQ ID NO: 16 - DNA, 33 bp, Artificial Sequence
  Description of Artificial Sequence: Synthetic primer
ggcagagacccctgcaacaacctggaagccttg

SEQ ID NO: 17 - DNA, 33 bp, Artificial Sequence
  Description of Artificial Sequence: Synthetic primer
caaggcttccaggttgttgcaggggtctctgcc

SEQ ID NO: 18 - DNA, 36 bp, Artificial Sequence
  Description of Artificial Sequence: Synthetic primer
ggcagagaccccaagaactgcctggaagccttggag

SEQ ID NO: 19 - DNA, 36 bp, Artificial Sequence
  Description of Artificial Sequence: Synthetic primer
ctccaaggcttccaggcagttcttggggtctctgcc

SEQ ID NO: 20 - DNA, 40 bp, Artificial Sequence
  Description of Artificial Sequence: Synthetic primer
catcagcaggtcgcactgcaaggatcactacatcttttac

SEQ ID NO: 21 - DNA, 40 bp, Artificial Sequence
  Description of Artificial Sequence: Synthetic primer
gtaaaagatgtagtgatccttgcagtgcgacctgctgatg

SEQ ID NO: 22 - protein, 152 aa, Artificial Sequence
  Description of Artificial Sequence: Synthetic polypeptide
ASDEEIQDVSGTWYLKAMTVDTQMPLSLYVSVTPICLTTLEGGSLEATVTLNQIGRSQEVRAVLEKTDEPGKYTAYGGAHVAYISRSHVKDHYIFYSEGDTWGGPVPGVWLVGRDPKNNLEALEDFEKAAGARGLSTESILIPRQSETSSPG

SEQ ID NO: 23 - protein, 152 aa, Artificial Sequence
  Description of Artificial Sequence: Synthetic polypeptide
ASDEEIQDVSGTWYLKAMTVDTQMPLSLYVSVTPITLTTLEGGSLEATVTLNQIGRSQEVRAVLEKTDEPGKYTAYGGAHVAYISRSHVKDHYIFYSEGDTWGGPVPGVWLVGRDPKNCLEALEDFEKAAGARGLSTESILIPRQSETSSPG

SEQ ID NO: 24 - protein, 152 aa, Artificial Sequence
  Description of Artificial Sequence: Synthetic polypeptide
ASDEEIQDVSGTWYLKAMTVDTQMPLSLYVSVTPITLTTLEGGSLEATVTLNQIGRSQEVRAVLEKTDEPGKYTAYGGAHVAYISRSHVKCHYIFYSEGDTWGGPVPGVWLVGRDPKNNLEALEDFEKAAGARGLSTESILIPRQSETSSPG

SEQ ID NO: 25 - protein, 152 aa, Artificial Sequence
  Description of Artificial Sequence: Synthetic polypeptide
ASDEEIQDVSGTWYLKAMTVDTQMPLSLYVSVTPITLTTLEGGSLEATVTLNQIGRSQEVRAVLEKTDEPGKYTAYGGAHVAYISCSHVKDHYIFYSEGDTWGGPVPGVWLVGRDPKNNLEALEDFEKAAGARGLSTESILIPRQSETSSPG

SEQ ID NO: 26 - protein, 152 aa, Artificial Sequence
  Description of Artificial Sequence: Synthetic polypeptide
ASDEEIQDVSGTWYLKAMTVDTQMPLSLYVSVTPITLTTLEGGSLEATVTLNQIGRSQEVRAVLEKTDEPGKYTAYGGAHVAYISRSHVKDHYIFYSEGDTWGGPVPGVWLVGRDPCNNLEALEDFEKAAGARGLSTESILIPRQSETSSPG

SEQ ID NO: 27 - DNA, 39 bp, Artificial Sequence
  Description of Artificial Sequence: Synthetic primer
  modified_base (22)..(23): a, c, g, t, unknown or other
gccatgacggtggacacgcagnnsccgctgagcctctac

SEQ ID NO: 28 - DNA, 33 bp, Artificial Sequence
  Description of Artificial Sequence: Synthetic primer
  modified_base (17)..(18): a, c, g, t, unknown or other
cagggtcgtgagggtsnngggtgtcaccgagac

SEQ ID NO: 29 - DNA, 57 bp, Artificial Sequence
  Description of Artificial Sequence: Synthetic primer
  modified_base (19)..(20): a, c, g, t, unknown or other
  modified_base (28)..(29): a, c, g, t, unknown or other
  modified_base (37)..(38): a, c, g, t, unknown or other
gggggcaacctggaagccnnsgtcaccnnsaaccagnnsggccggtcccaggaggtg

SEQ ID NO: 30 - DNA, 57 bp, Artificial Sequence
  Description of Artificial Sequence: Synthetic primer
  modified_base (38)..(39): a, c, g, t, unknown or other
gtattttcccggctcatcagttttctccaggacggcsnncacctcctgggaccggcc

SEQ ID NO: 31 - DNA, 41 bp, Artificial Sequence
  Description of Artificial Sequence: Synthetic primer
  modified_base (21)..(22): a, c, g, t, unknown or other
gtgctcacgtggcatacatcnnsaggtcgcacgtgaaggac

SEQ ID NO: 32 - protein, 111 aa, Artificial Sequence
  Description of Artificial Sequence: Synthetic polypeptide
ASDEEIQDVSGTWYLKAMTVDTQDPLSLYVSVTPITLTTLEGGNLEAMVTLNQVGRSQEVLAVLEKTDEPGKYTAYGGAHVAYIQRSHVKDHYIFYSEGDTWGGPVPGVWL

SEQ ID NO: 33 - protein, 111 aa, Artificial Sequence
  Description of Artificial Sequence: Synthetic polypeptide
ASDEEIQDVSGTWYLKAMTVDTQDPLSLYVSVTPLTLTTLEGGNLEAMVTLNQVGRSQEVLAVLEKTDEPGKYTAYGGAHVAYITRSHVKDHYIFYSEGDTWGGPVPGVWL

SEQ ID NO: 34 - protein, 111 aa, Artificial Sequence
  Description of Artificial Sequence: Synthetic polypeptide
ASDEEIQDVSGTWYLKAMTVDTQDPLSLYVSVSPITLTTLEGGNLEATVTLNQIGRSQEVLAVLEKTDEPGKYTAYGGAHVAYIHRSHVKDHYIFYSEGDTWGGPVPGVWL

SEQ ID NO: 35 - protein, 152 aa, Artificial Sequence
  Description of Artificial Sequence: Synthetic polypeptide
ASDEEIQDVSGTWYLKAMTVDTQDPLSLYVSVTPITLTTLEGGNLEAMVTLNQVGRSQEVLAVLEKTDEPGKYTAYGGAHVAYIQRSHVKDHYIFYSEGDTWGGPVPGVWLVGRDPKNCLEALEDFEKAAGARGLSTESILIPRQSETSSPG

SEQ ID NO: 36 - protein, 158 aa, Homo sapiens
HHLLASDEEIQDVSGTWYLKAMTVDREFPEMNLESVTPMTLTTLEGGNLEAKVTMLISGRSQEVKAVLEKTDEPGKYTADGGKHVAYIIRSHVKDHYIFYSEGELHGKPVPGVWLVGRDPKNNLEALEDFEKAAGARGLSTESILIPRQSETSSPGSD

SEQ ID NO: 37 - protein, 158 aa, Artificial Sequence
  Description of Artificial Sequence: Synthetic polypeptide
ASDEEIQDVSGTWYLKAMTVDTQTPLSLYVSVTPITLTTLEGGNLEAEVTLNQVGRSQEVRAVLEKTDEPGKYTAYGGAHVAYIIRSHVKDHYIFYSEGDTWGGPVPGVWLVGRDPKNNLEALEDFEKAAGARGLSTESILIPRQSETSSPGHHHHHH

SEQ ID NO: 38 - protein, 158 aa, Artificial Sequence
  Description of Artificial Sequence: Synthetic polypeptide
ASDEEIQDVSGTWYLKAMTVDTQMPLSLYVSVTPITLTTLEGGSLEATVTLNQIGRSQEVRAVLEKTDEPGKYTAYGGAHVAYISRSHVKDHYIFYSEGDTWGGPVPGVWLVGRDPKNNLEALEDFEKAAGARGLSTESILIPRQSETSSPGHHHHHH

SEQ ID NO: 39 - protein, 158 aa, Artificial Sequence
  Description of Artificial Sequence: Synthetic polypeptide
ASDEEIQDVSGTWYLKAMTVDTQDPLSLYVSVTPITLTTLEGGNLEAMVTLNQVGRSQEVLAVLEKTDEPGKYTAYGGAHVAYIQRSHVKDHYIFYSEGDTWGGPVPGVWLVGRDPKNNLEALEDFEKAAGARGLSTESILIPRQSETSSPGHHHHHH

SEQ ID NO: 40 - protein, 158 aa, Artificial Sequence
  Description of Artificial Sequence: Synthetic polypeptide
ASDEEIQDVSGTWYLKAMTVDTQDPLSLYVSVTPLTLTTLEGGNLEAMVTLNQVGRSQEVLAVLEKTDEPGKYTAYGGAHVAYITRSHVKDHYIFYSEGDTWGGPVPGVWLVGRDPKNNLEALEDFEKAAGARGLSTESILIPRQSETSSPGHHHHHH

SEQ ID NO: 41 - protein, 158 aa, Artificial Sequence
  Description of Artificial Sequence: Synthetic polypeptide
ASDEEIQDVSGTWYLKAMTVDTQDPLSLYVSVSPITLTTLEGGNLEATVTLNQIGRSQEVLAVLEKTDEPGKYTAYGGAHVAYIHRSHVKDHYIFYSEGDTWGGPVPGVWLVGRDPKNNLEALEDFEKAAGARGLSTESILIPRQSETSSPGHHHHHH

SEQ ID NO: 42 - protein, 6 aa, Artificial Sequence
  Description of Artificial Sequence: Synthetic 6xHis tag
HHHHHH

(19) United States
(12) Patent Application Publication
(10) Pub. No.: US 2009/0274623 A1
(43) Pub. Date: Nov. 5, 2009

(54) IN VIVO IMAGING AGENTS FOR MET RECEPTOR TYROSINE KINASE

(75) Inventors: Clifford L. Smith, Hertfordshire (GB); Faisal Ahmed Syud, Clifton Park, NY (US); Brian Duh-Lan Lee, Rexford, NY (US); Matthew Sam Morrison, Buckinghamshire (GB); Michael Ernest Marino, Clifton Park, NY (US); Jason William Castle, Esperance, NY (US); Paul Schaffer, Clifton Park, NY (US); Gabriele Matschiner, Munchen (DE); Andreas Hohlbaum, Paunzhausen (DE); Martin Huelsmeyer, Wolfersdorf (DE); Stefan Trentmann, Allershausen (DE)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: GENERAL ELECTRIC COMPANY, Schenectady, NY (US)

(21) Appl. No.: 12/022,526

(22) Filed: Jan. 30, 2008

(51) Int. Cl. A61K 51/08; A61K 49/00; A61K 49/14

(52) U.S. Cl. 424/1.69; 424/9.1; 424/9.34; 424/9.6

(57) ABSTRACT

Provided are in vivo imaging agents comprising muteins of hTLc having detectable binding affinity for c-Met or a domain thereof, coupled to a signal generator, and the mutein coupled to a signal generator is disposed in a pharmaceutically acceptable carrier. Also provided are diagnostic methods using in vivo imaging agents comprising muteins of hTLc having detectable binding affinity for c-Met or a domain thereof.

### FIELD

In general, the field of invention relates to in vivo imaging agents comprising muteins of tear lipocalins that bind to human Met receptor tyrosine kinase (c-Met). More specifically, the field of invention relates in vitro and in vivo methods using tear lipocalins that bind to human c-Met.

### BACKGROUND

The Met receptor tyrosine kinase was first identified as the product of a human oncogene, Tpr-Met (Park et al., Proc. Natl. Acad. Sci., USA, Vol. 84, pages 6379-6383, 1987). The ligand for c-Met was identified as hepatocyte growth factor (HGF). HGF was originally identified as a mitogen for hepatocytes in culture. HGF is identical to scatter factor (SF), a fibroblast-derived factor that promotes dispersal of sheets of epithelial cells, as well as branching tubulogenesis of epithelia grown in three-dimensional cultures. HGF/SF is a unique growth factor that elicits multiple cellular responses including mitogenesis, cell motility and morphogenesis.

c-Met is predominantly expressed epithelial cells, but is also expressed in endothelial cells, neural cells, hepatocytes, hematopoietic cells, and melanocytes. c-Met activation plays a key role in cellular physiology: mitogenesis, motogenesis, and morphogenesis. Activated c-Met activates Grb2 (growth factor receptor bound protein 2) and Gab 1 (growth factor receptor bound protein 2 associated binder 1). Grb2, in turn, activates a number of kinase pathways, including the pathway from Ras to Raf to Mek and to MAPK (mitogen-activated protein kinase). Gab 1 activates PI3K (phosphoinositide 3 kinase), which activates STAT3 (signal transducer and activator of transcription). c-Met activation also induces activation of beta catenin, a key component of the wnt pathway, which translocates into the nucleus and participates in transcription regulation.

The HGF/c-Met pathway plays an important role in the development of cancer. First through the activation of key oncogenic pathways (Ras, PI3K/STAT3, and beta catenin), secondly through endothelial cell proliferation (neoangiogenesis), and thirdly through increased protease production and cell dissociation leading to metastasis.

Various new therapeutic approaches are aimed at the HGF/c-Met pathway. These approaches include anti-HGF monoclonal antibodies such the humanized form AV299 of AVEO or a fully human antibody named AMB102 one from Amgen (AMG102). Another approach is the use of truncated variants of c-Met that act as decoys. One such example is the truncated version called CGEN241 from COMPUGEN. Also protein kinase inhibitors that block c-Met induced pathways are used for therapeutic purpose.

However, it is still desirable to have further compounds available that bind c-Met that may be used for therapeutic and/or diagnostic purposes.

### SUMMARY OF THE INVENTION

The present invention relates to imaging agents comprising mutein of human tear lipocalin (hTLc) having detectable binding affinity to the human Met receptor tyrosine kinase (c-Met) or a domain or fragments thereof and imaging methods using such agents.

Provided herein are in vivo imaging agents comprising a mutein of hTLc having detectable binding affinity for c-Met or a domain thereof, wherein the mutein comprises amino acid replacements for at least one sequence position corresponding to sequence positions 26-34, 56-58, 80, 83, 104-106, and 108 of hTLc of SEQ. ID. NO:36, the mutein is coupled to a signal generator, and the mutein coupled to a signal generator is disposed in a pharmaceutically acceptable carrier.

The mutein component of the in vivo imaging agent including a tear lipocalin mutein may comprise at least 2, 3, 4, 5, 6, 8, 10, 12, 14, 16 or 18 mutated amino acid residues at the sequence positions 26-34, 56-58, 80, 83, 104-106, or 108 of SEQ. ID. NO:36.

In some embodiments, the mutein component of the in vivo imaging agent including a tear lipocalin mutein may also comprise mutated amino acid residues at sequence positions 26, 27, 28, 30, 31, 32, 33, 34, 56, 57, 58, 80, 83, 104, 105, 106, or 108 of SEQ. ID. NO: 36.

In other embodiments, the mutein component of the in vivo imaging agent including a tear lipocalin mutein may also comprise mutated amino acid residues at least one of the amino acid substitutions Cys 61→Ser, Cys 101→Ser, or Cys 153→Ser of SEQ. ID. NO: 36.

In yet other embodiments, the mutein component of the in vivo imaging agent including a tear lipocalin mutein may also comprise mutated amino acid residues wherein the mutein comprises at least one additional amino acid substitution selected from Arg 111→Pro or Lys 114→Trp.

In some specific embodiments, mutein component of the in vivo imaging agent including a tear lipocalin mutein may also comprise amino acid sequence of any one of SEQ. ID. NOs: 1, 4-9, 22-26-32-35 and 37-41.

The signal generator may comprise a radionuclide (e.g., ¹¹C, ¹⁸F, ⁶⁷Ga, ⁶⁸Ga, ^{94m}Tc, ^{99m}Tc, ⁶⁴Cu, ⁶⁷Cu, ¹²³I, or ¹²⁴I), a paramagnetic ion, a chemiluminescent agent (e.g., green fluorescent protein, yellow fluorescent protein, or luciferin), or a fluorophore (e.g., a cyanine dye or a quantum dot).

The radionuclide is 18F, 123I, or 124I may be attached to the mutein via an aminoxy group and the aminoxy group may be bound to a thiol-containing amino acid residue (e.g., a cysteine) present in the mutein.

In some embodiments, the radionuclide is ^{94m}Tc or ^{99m}Tc that is attached to the mutein via an imidazole-containing tag selected from tris-histidine or hexa-histidine. In some alternative embodiments, the radionuclide may be attached to the mutein via a linker selected from a bis amine-oxime (e.g., cPn216) or hydrazine nicotinic acid (e.g., HYNIC).

In some embodiments, the linker may be bound to a thiol-containing amino acid residue present in the mutein.

In some embodiments, the radionuclide is selected from ⁶⁷Ga, ⁶⁸Ga, ⁶⁴Cu, or ⁶⁷Cu that is attached to the mutein via a chelator selected from NOTA, DOTA, or DTPA. The chelator may be bound to a thiol-containing amino acid residue present in the mutein.

In some embodiments, the fluorescent agent is bound to a thiol-containing amino acid residue present in the mutein.

Also provided herein are methods of detecting a cell proliferative disorder in a mammalian subject comprising: a) administering an in vivo imaging agent comprising a mutein of hTLc having detectable binding affinity for c-Met or a domain thereof, wherein the mutein comprises amino acid replacements for at least one sequence position corresponding to sequence positions 26-34, 56-58, 80, 83, 104-106, and 108 of hTLc of SEQ. ID. NO:36, the mutein is coupled to a signal generator, and the mutein coupled to a signal generator is disposed in a pharmaceutically acceptable carrier; and ( ) observing the signal produced by the in vivo agent.

In some embodiments, the cell proliferative disorder is selected from liver cancer, colon cancer, colorectal cancer, hepatocellular carcinoma, papillary renal carcinoma, head and neck squamous cell carcinoma (HNSC), lymph nodes metastases of head and neck, or squamous carcinoma.

In embodiments wherein the signal generator is a radionuclide selected from ¹¹C, ¹⁸F, ⁶⁸Ga, ¹²⁴I, ⁶⁴Cu, or ^{94m}Tc, the signal may be observed using positron emission tomography.

In embodiments wherein the signal generator is a radionuclide selected from ^{99m}Tc, ⁶⁷Ga, ¹²³I, or ⁶⁷Cu, the signal is observed using single photon emission computed tomography.

In embodiments wherein the paramagnetic ion is selected from 157Gd, 55Mn, 162 Dy, 52Cr, or 56Fe and the signal is observed using magnetic resonance.

In embodiments wherein signal generator is a fluorophore (e.g., a cyanine dye or a quantum dot), the signal may be observed using optical imaging.

In some embodiments, the mutein may be administered to the mammalian subject parenterally via intracutaneous, subcutaneous, intramuscular, intratracheal, intranasal, intravitreal, or intravenous injection or infusion.

### FIGURES

Practice will be still more fully understood from the following examples, which are presented herein for illustration only and should not be construed as limiting the invention in any way.

FIG. 1 shows a map of phagemid vector pTLPC59, with FIG. 1a showing a schematic presentation of the regulatory elements of the vector and FIG. 1b representing a schematic enlargement of the gene construct of the tear lipocalin muteins that is used for expressing the naïve library.

FIG. 2 shows a map of the expression vector pTLPC 10.

FIG. 3 shows the polypeptide sequences of the tear lipocalin muteins S225.4-K24 (SEQ ID NO:1) in alignment with the polypeptide sequences of wild type human tear lipocalin (a truncation is provided as SEQ ID NO:36) beginning at residue 5 of the human tear lipocalin. The naming conventions used herein for substitutions refer to the sequence provided in SEQ. ID. NO: 36. Accordingly, a substitution of Cys for Asn at position 123 of SEQ. ID. NO: 36 is shown in SEQ. ID. NO: 35 at position 119.

FIG. 4 shows the results of affinity screening via ELISA for muteins with affinity for c-Met.

FIG. 5 shows an alignment of the polypeptide sequences of the tear lipocalin muteins S225.4-K24 (SEQ ID NO: 1), and S244.2-H08, S244.2-L01, S244.4-N05, S244.5-J05, S244.8-I20, S244.8-I07 (SEQ ID NOs.: 4-9),

FIG. 6 shows BIAcore measurements of the binding of an hTLc mutein (S244.2-H08; SEQ ID NO:4) to c-Met.

FIG. 7 shows the result of an affinity assessment of the c-Met binding muteins S244.2-H08, S244.2-L01, S244.4-N05, S244.5-J05, S244.8-120, S244.8-107 (SEQ ID NOs.: 4-9) in a cellular context on HT29 cells.

FIG. 8 shows the results method of affinity screening via ELISA for muteins with affinity for c-Met.

FIG. 9 shows an alignment of the polypeptide sequences of the tear lipocalin muteins S225.4-K24 (SEQ ID. NO:1), S244.2-H08 (SEQ ID. NO:4), S261.1-L12, S261.1-J01, and S261.1-L17 (SEQ ID. NOs:32-34) with hTLc (SEQ. ID. NO:36).

FIG. 10 shows a map of the expression vector pTLPC 47.

FIG. 11 shows the result of an affinity assessment of the c-Met binding muteins S261.1-L12, S261.1-J01, and S261.1-L17 (SEQ ID. NOs:32-34) in a cellular context on HT29 cells.

FIG. 12 shows competition ELISA measurements of the binding of an hTLc mutein (S261.1-L17; SEQ ID NO:34) to c-Met.

FIG. 13 shows the result of an affinity assessment of the c-Met binding mutein S261.1-L12_C123 (SEQ ID NO:35) in a cellular context on HT29 cells.

FIG. 14 shows the results of the pH stability test of the tear lipocalin mutein S261.1-J01 (SEQ ID NO:33).

FIG. 15 shows an alignment of the polypeptide sequences of further tear lipocalin muteins (in which further single mutations have been introduced) together with their KD value for the binding to c-Met.

FIG. 16 shows the sonogram showing the binding of HIS-6-S244.2-H08 (SEQ. ID. NO: 38), HIS-6-S244.2-L01 (SEQ. ID. NO:37), and hTLc (SEQ ID NO:36) for c-Met extracellular domain measured using surface plasmon resonance (SPR).

FIG. 17 shows in vitro binding of HIS-6-S244.2-H08 (SEQ. ID. NO: 38), HIS-6-S244.2-L01 (SEQ. ID. NO:37) to two cell lines HT29 (c-Met positive) and C6 (c-Met negative).

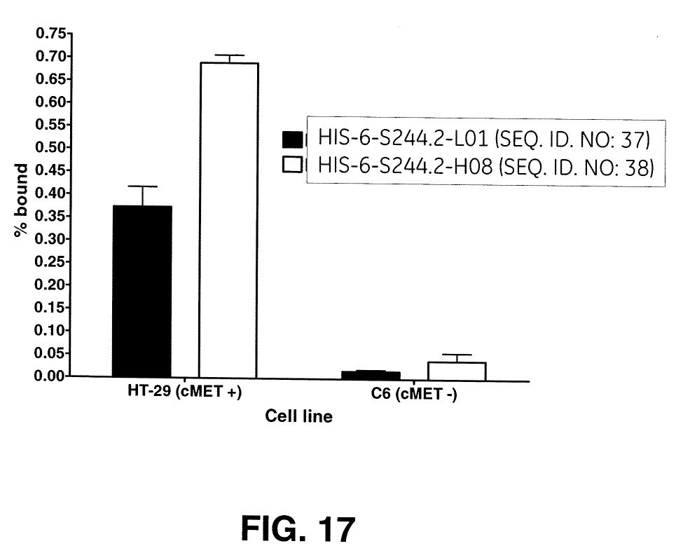

FIG. 18 shows the clearance profile of HIS-6-S244.2-L01 (SEQ. ID. NO:37) in blood, bladder, liver and kidney.

FIG. 19 shows HIS-6-S244.2-H08 (SEQ. ID. NO: 38), HIS-6-S244.2-L01 (SEQ. ID. NO:37) and hTLc (SEQ ID NO:36) uptake in tumor tissue over two hours.

FIG. 20 shows tumor to blood ratio of HIS-6-S244.2-H08 (SEQ. ID. NO: 38), HIS-6-S244.2-L01 (SEQ. ID. NO:37), and hTLc (SEQ ID NO:36) over two hours.

FIG. 21 shows a blocking study that compares labeled HIS-6-S244.2-L01 (SEQ. ID. NO:37) (^{99m}Tc-L01) against combined labeled and unlabeled HIS-6-S244.2-L01 (SEQ. ID. NO:37) in blood, tumor, liver, kidney, and spleen.

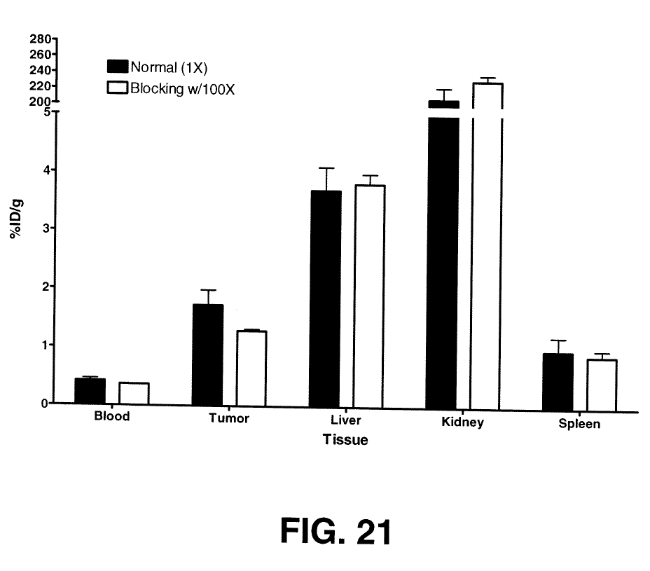

FIG. 22 shows in vivo binding of labeled HIS-6-S244.2-H08 (SEQ. ID. NO: 38), HIS-6-S244.2-L01 (SEQ. ID. NO:37) HIS-6-S2261.1-L12 (SEQ. ID. NO: 39), HIS-6-S2261.1-L17 (SEQ. ID. NO: 41), HIS-6-S2261.1-J01 (SEQ. ID. NO: 40), and hTLc (SEQ ID NO:36) levels at 2 hours post-injection using HT29 tumor-bearing CD-1 nude mice.

FIG. 23 shows S2261.1-L12_C123 (SEQ. ID. NO: 35) —HYNIC levels following administration to HT29 tumor-bearing CD-1 nude mice over four hours.

FIG. 24 shows the comparison of S2261.1-L12_C123 (SEQ. ID. NO: 35) —HYNIC HYNIC levels to hTLc (SEQ ID NO:36) tumor-to-blood ratios following administration to HT29 tumor-bearing CD-1 nude mice over four hours.

FIG. 25 shows S2261.1-L12_C123 (SEQ. ID. NO:35) —HYNIC clearance from blood, tumor, liver, and kidney over four hours.

FIG. 26 shows in vivo clearance of optically labeled S2261.1-L12_C123 (SEQ. ID. NO:35) and optically labeled hTLc (SEQ ID NO:36) over 24 hours.

### DETAILED DESCRIPTION

The following detailed description is exemplary and not intended to limit the invention of the application and uses. Furthermore, there is no intention to be limited by any theory presented in the preceding background of the following detailed description of the figures.

To more clearly and concisely describe and point out the subject matter of the claimed invention, the following definitions are provided for specific terms that are used in the following description and the claims appended hereto.

As used herein the term “binding” refers to the ability of a binder to preferentially bind to target with an affinity that is at least two-fold greater than its affinity for binding to a non-specific target (e.g., BSA or casein) other than the predetermined target or a closely-related target. The binders provided herein bind their respective targets with an affinity with a KD value less than about 1×10⁶ M⁻¹, more preferably less than about 1×10⁷ M⁻¹, and most preferably less than about 1×10⁸ M⁻¹.

As used herein, the phrase “blood half-life” refers to the time required for the plasma concentration of an agent to decline by one-half when elimination is first-order or pseudo-first order. In the case of multiple decay phases, the term “blood half life” refers to either the apparent half-life (if the decay half-lives for different phases are similar) or the dominant half-life (that accounting for the bulk of the clearance) if the different half-lives are dissimilar.

The phrase “detecting a cancer” or “diagnosing a cancer” refers to determining the presence or absence of cancer or a precancerous condition in an animal. “Detecting a cancer” also may refer to obtaining indirect evidence regarding the likelihood of the presence of precancerous or cancerous cells in the animal or assessing the predisposition of a patient to the development of a cancer. Detecting a cancer may be accomplished using the methods of this invention alone, in combination with other methods, or in light of other information regarding the state of health of the animal.

As used herein, the term “fluorophore” refers to a chemical compound, which when excited by exposure to a particular wavelength of light, emits light (at a different wavelength. Fluorophores may be described in terms of their emission profile, or “color.” Green fluorophores (for example Cy3, FITC, and Oregon Green) may be characterized by their emission at wavelengths generally in the range of 515-540 nanometers. Red fluorophores (for example Texas Red, Cy5, and tetramethylrhodamine) may be characterized by their emission at wavelengths generally in the range of 590-690 nanometers.

The term “hTLc fragment” as used herein, relates to proteins or peptides derived from full-length mature hTLc that are N-terminally and/or C-terminally shortened, for example lacking at least one of the N-terminal and/or C-terminal amino acids. Such fragments comprise preferably at least 10, more preferably 20, most preferably 30 or more consecutive amino acids of the primary sequence of mature hTLc and are usually detectable in an immunoassay of mature hTLc.

The term “fusion protein” as used herein also comprises lipocalin muteins according to the invention containing a signal sequence. Signal sequences at the N-terminus of a polypeptide direct this polypeptide to a specific cellular compartment, for example the periplasm of E. coli or the endoplasmic reticulum of eukaryotic cells. A preferred signal sequence for secretion a polypeptide into the periplasm of E. coli is the OmpA-signal sequence.

The term “human tear lipocalin” and the abbreviation “hTLc” as used herein refer to the mature hTLc protein deposited in the SWISS-PROT Data Bank at Accession Number P31025 (a truncation of which is provided as SEQ. ID. NO:36). With regard to the nucleic acids, the term refers to any nucleic acid that encodes for the protein sequence deposited as Accession Number P31025.

As used herein, with regard to the introduction the disclosed agents to a body, the phrase “in vivo” refers to methods for directly administering the disclosed agents to the subject's body under conditions where the c-Met mutein is able to interact with and bind to endogenous c-Met. The agents of the present invention or their pharmaceutically acceptable salts may be administered to the subject in a variety of forms adapted to the chosen route of administration.

The term “mutagenesis” as used herein means that the experimental conditions are chosen such that the amino acid naturally occurring at a given sequence position of hTLc may be substituted by at least one amino acid that is not present at this specific position in the respective natural polypeptide sequence. The term “mutagenesis” also includes the (additional) modification of the length of sequence segments by deletion or insertion of one or more amino acids. Thus, it is within the scope that, for example, one amino acid at a chosen sequence position is replaced by a stretch of three random mutations, leading to an insertion of two amino acid residues compared to the length of the respective segment of the wild type protein. Such an insertion of deletion may be introduced independently from each other in any of the peptide segments that may be subjected to mutagenesis in the invention. In one exemplary embodiment, an insertion of several mutations may be introduced into the loop AB of the chosen lipocalin scaffold (cf. International Patent Application WO 2005/019256, which is incorporated by reference its entirety herein). The phrase “random mutagenesis” means that no predetermined single amino acid (mutation) is present at a certain sequence position but that at least two amino acids may be incorporated with a certain probability at a predefined sequence position during mutagenesis.

A nucleic acid molecule, such as DNA, is “operably linked” to a regulatory sequence elements and the sequence to be expressed are connected in a way that enables gene expression. The precise nature of the regulatory regions necessary for gene expression may vary among species, but in general these regions comprise a promoter which, in prokaryotes, contains both the promoter per se, i.e. DNA elements directing the initiation of transcription, as well as DNA elements which, when transcribed into RNA, will signal the initiation of translation. Such promoter regions normally include 5′ non-coding sequences involved in initiation of transcription and translation, such as the -35/-0 boxes and the Shine-Dalgarno element in prokaryotes or the TATA box, CAAT sequences, and 5′-capping elements in eukaryotes. These regions may also include enhancer or repressor elements as well as translated signal and leader sequences for targeting the native polypeptide to a specific compartment of a host cell.

As used herein the term “paramagnetic metal ion”, “paramagnetic ion” or “metal ion” refers to a metal ion that is magnetized parallel or antiparallel to a magnetic field to an extent proportional to the field. Generally, these are metal ions that have unpaired electrons. Examples of suitable paramagnetic metal ions, include, but are not limited to, Gd⁺³Fe⁺³, Mn⁺², Yt⁺³, Dy⁺³, and Cr⁺³.

As used herein the term “pharmaceutically-acceptable carriers” refers to those carriers which are, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible, of the agents.

As used herein, the term “signal generator” refers to a molecule capable of providing a detectable signal using one or more detection techniques (e.g., spectrometry, calorimetry, spectroscopy, or visual inspection). Suitable examples of a detectable signal may include an optical signal, and electrical signal, or a radioactive signal. Examples of signal generators useful in the methods include, for example, a chromophore, a fluorophore, a Raman-active tag, a radioactive label, an enzyme, an enzyme substrate, or combinations thereof. Suitable radioisotopes may include ³H, ¹¹C, ¹⁴C, ¹⁸F, ³²P, ³⁵S, ¹²³I, ¹²⁵I, ¹³¹I, ⁵¹Cr, ³⁶Cl, ⁵⁷Co, ⁵⁹Fe, ⁷⁵Se, and ¹⁵²Eu. Isotopes of halogens (such as chlorine, fluorine, bromine and iodine), and metals including technetium, yttrium, rhenium, and indium are also useful labels. Typical examples of metallic ions that may be used as signal generators include ^{99m}Tc, ¹²³I, ¹¹¹In, ¹³¹I, ⁹⁷Ru, ⁶⁷Cu, ⁶⁷Ga, ¹²⁵I, ⁶⁸Ga, ⁷²As, ⁸⁹Zr, and ²⁰¹Tl.

The term “variant” as used herein relates to derivatives of a protein or peptide that comprise modifications of the amino acid sequence, for example by substitution, deletion, insertion or chemical modification. Preferably, such modifications do not reduce the functionality of the protein or peptide. Such variants include proteins, wherein one or more amino acids have been replaced by their respective D-stereoisomers or by amino acids other than the naturally occurring 20 amino acids, such as, for example, ornithine, hydroxyproline, citrulline, homoserine, hydroxylysine, or norvaline. However, such substitutions may also be conservative such that an amino acid residue is replaced with a chemically similar amino acid residue. Examples of conservative substitutions are the replacements among the members of the following groups: (1) alanine, serine, and threonine; (2) aspartic acid and glutamic acid; (3) asparagine and glutamine; (4) arginine and lysine; (5) isoleucine, leucine, methionine, and valine; and (6) phenylalanine, tyrosine, and tryptophan.

Unless otherwise indicated, all numbers expressing quantities of ingredients, properties such as molecular weight, reaction conditions, so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least each numerical parameter should at least be construed in light of the number of reported significant digits and by applying ordinary rounding techniques.

### Mutein Production

The coding sequence of hTLc (SEQ. ID. NO:36) is used as a starting point for the mutagenesis of the peptide segments selected in the present invention. A commonly used technique is the introduction of mutations by means of PCR (polymerase chain reaction) using mixtures of synthetic oligonucleotides, which bear a degenerate base composition at the desired sequence positions. For example, use of the codon NNK or NNS (wherein N=adenine, guanine or cytosine or thymine; K=guanine or thymine; S=adenine or cytosine) allows incorporation of all 20 amino acids plus the amber stop codon during mutagenesis, whereas the codon VVS limits the number of possibly incorporated amino acids to 12, since it excludes the amino acids Cys, Ile, Leu, Met, Phe, Trp, Tyr, Val from being incorporated into the selected position of the polypeptide sequence; use of the codon NMS (wherein M=adenine or cytosine), for example, restricts the number of possible amino acids to 11 at a selected sequence position since it excludes the amino acids Arg, Cys, Gly, Ile, Leu, Met, Phe, Trp, Val from being incorporated at a selected sequence position. In this respect it is noted that codons for other amino acids (than the regular 20 naturally occurring amino acids) such as selenocysteine or pyrrolysine may also be incorporated into a nucleic acid of a mutein. It is also possible, as described by Wang, L., et al. (2001) Science 292, 498-500, or Wang, L., and Schultz, P. G. (2002) Chem. Comm. 1, 1-11, to use artificial codons such as UAG which are usually recognized as stop codons to insert other unusual amino acids, for example o-methyl-L-tyrosine or p-aminophenylalanine.

The use of nucleotide building blocks with reduced base pair specificity, as for example inosine, 8-oxo-2′deoxyguanosine or 6(2-deoxy-β-D-ribofuranosyl)-3,4-dihydro-8H-pyrimindo-1,2-oxazine-7-one (Zaccolo et al. (1996) J. Mol. Biol. 255, 589-603), is another option for the introduction of mutations into a chosen sequence segment.

A further possibility is the triplet-mutagenesis. This method uses mixtures of different nucleotide triplets, each of which codes for one amino acid, for incorporation into the coding sequence (Virnekas B, Ge L, Pluckthun A, Schneider K C, Wellnhofer G, Moroney S E. (1994) Trinucleotide phosphoramidites: ideal reagents for the synthesis of mixed oligonucleotides for random mutagenesis. Nucleic Acids Res. 22, 5600-5607).

One strategy for introducing mutations in the selected regions of the respective polypeptides is based on the use of four oligonucleotides, each of which is partially derived from one of the corresponding sequence segments to be mutated. When synthesizing these oligonucleotides, a person skilled in the art may employ mixtures of nucleic acid building blocks for the synthesis of those nucleotide triplets which correspond to the amino acid positions to be mutated so that codons encoding all natural amino acids randomly arise, which at last results in the generation of a lipocalin peptide library. For example, the first oligonucleotide corresponds in its sequence (apart from the mutated positions) to the coding strand for the peptide segment to be mutated at the most N-terminal position of the lipocalin polypeptide. Accordingly, the second oligonucleotide corresponds to the non-coding strand for the second sequence segment following in the polypeptide sequence. The third oligonucleotide corresponds in turn to the coding strand for the corresponding third sequence segment. Finally, the fourth oligonucleotide corresponds to the non-coding strand for the fourth sequence segment. A polymerase chain reaction may be performed with the respective first and second oligonucleotide and separately, if necessary, with the respective third and fourth oligonucleotide.

The amplification products of both of these reactions may be combined by various methods into a single nucleic acid comprising the sequence from the first to the fourth sequence segments, in which mutations have been introduced at the selected positions. To this end, both of the products may for example be subjected to a new polymerase chain reaction using flanking oligonucleotides as well as one or more mediator nucleic acid molecules, which contribute the sequence between the second and the third sequence segment.

The nucleic acid molecules may be connected by ligation with the missing 5′- and 3′-sequences of a nucleic acid encoding a lipocalin polypeptide and/or the vector, and may be expressed in host organism. A multitude of established procedures are available for ligation and cloning (Sambrook, J. et al. (1989), supra). For example, recognition sequences for restriction endonucleases also present in the sequence of the cloning vector may be engineered into the sequence of the synthetic oligonucleotides. Thus, after amplification of the respective PCR product and enzymatic cleavage the resulting fragment may be easily cloned using the corresponding recognition sequences.

Longer sequence segments within the gene coding for the protein selected for mutagenesis may also be subjected to random mutagenesis via known methods, for example by use of the polymerase chain reaction under conditions of increased error rate, by chemical mutagenesis or by using bacterial mutator strains. Such methods may also be used for further optimization of the target affinity or specificity of a lipocalin mutein. Mutations possibly occurring outside the segments of experimental mutagenesis are often tolerated or may even prove to be advantageous, for example if they contribute to an improved folding efficiency or folding stability of the lipocalin mutein.

According to the method a mutein is obtained starting from a nucleic acid encoding hTLc. Such a nucleic acid is subjected to mutagenesis and introduced into a suitable bacterial or eukaryotic host organism by means of recombinant DNA technology. Obtaining a nucleic acid library of tear lipocalin may be carried out using any suitable technique that is known in the art for generating lipocalin muteins with antibody-like properties, i.e. muteins that have affinity towards a given target. Examples of such combinatorial methods are described in detail in the international patent applications WO 99/16873, WO 00/75308, WO 03/029471, WO 03/029462, WO 03/029463, WO 2005/019254, WO 2005/019255, WO 2005/019256, WO 2006/56464, or International patent application PCT/EP2007/057971 the disclosures of which are incorporated by reference herein. After expression of the nucleic acid sequences that were subjected to mutagenesis in an appropriate host, the clones carrying the genetic information for the plurality of respective lipocalin muteins, which bind a given target may be selected from the library obtained.

Well known techniques may be employed for the selection of these clones, such as phage display (reviewed in Kay, B. K. et al. (1996) supra; Lowman, H. B. (1997) supra or Rodi, D. J., and Makowski, L. (1999) supra), colony screening (reviewed in Pini, A. et al. (2002) Comb. Chem. High Throughput Screen. 5, 503-510), ribosome display (reviewed in Amstutz, P. et al. (2001) Curr. Opin. Biotechnol. 12, 400-405) or mRNA display as reported in Wilson, D. S. et al. (2001) Proc. Natl. Acad. Sci. USA 98, 3750-3755 or the methods specifically described in WO 99/16873, WO 00/75308, WO 03/029471, WO 03/029462, WO 03/029463, WO 2005/019254, WO 2005/019255, WO 2005/019256, WO 2006/56464 or International patent application PCT/EP2007/057971, the disclosures of which are incorporated by reference herein.

Generating c-Met Binding Muteins

For the generation of c-Met binding tear lipocalin muteins, any portion (for example, a fragment or single domain) of the extracellular domains of the human Met receptor tyrosine kinase (c-Met) or the entire extracellular domains (that comprises the N-terminal amino acid residues 1 methionine-threonine 932 of the mature entire receptor (Swiss Prot Database Accession No. P08581) may be contacted with the muteins that have been obtained from the expression of the (naïve) nucleic acid library that encodes these muteins. It is possible to use the commercially available extracellular domains that, for example are provided as residues 1-932 fused to an Fc region of a human IgG via a polypeptide linker, for example (R & D Systems, USA, catalog number 358-MT). Further examples of fragments of c-Met that may be used for obtaining muteins described here include, but are not limited to a fragment consisting of the residues 25 to 567 of c-Met as described in Stamos et al., The EMBO Journal Vol. 23, No. 12, 2004, pp. 2325-2335 that contain the seven SEMA Domains, or larger fragments that comprise residues 25 to 567. Fragments binding the SEMA domain may be used, if the tear lipocalin muteins are supposed to compete with binding of HGF to the SEMA domains. Such muteins may (but do not necessarily have to have, see examples) antagonists of HGF. It is also possible to use fragments such as the one comprising residues 568 to 932, if binding to the SEMA domains is to be avoided.

Screening may also be carried out using fragments or other domains such as the PSI domain or the IgG-like domains of c-Met. It is also possible to use for screening purposes, for example, the homolog of the common chimpanzees (pan troglodytes, 99% identity to human c-Met), the macaca homolog (macaca mulatta, 98% identity), the canine ortholog (canis familiaris, 88% identity), the mouse ortholog (Swiss Prot Database Accession A1A597, 87% identity) or the rat ortholog (rattus norvegicus, 86% identity) in place of the (extracellular domains of) human c-Met. Such an approach could for example be taken, if muteins having cross-reactivity between the human and the mouse or the rat ortholog (or extracellular domains, for example) would be desired. As it is clear from the above, it is possible to generate in the present invention muteins of tear lipocalin that may have an antagonist action in relation to HGF. Alternatively, the muteins may have a respective non-antagonistic binding mode.

In one embodiment, the selection is carried out under competitive conditions in which the muteins and the given non-natural ligand of hTLc (target) are brought in contact in the presence of an additional ligand such as HGF, which competes with binding of the muteins to the target. This additional ligand may be a physiological ligand of c-Met such as HGF or a non-physiological ligand of the c-Met such as an anti-c-Met antibody or a small molecule protein tyrosine kinase inhibitor that binds at least an overlapping or partly overlapping epitope to the epitope recognized by the muteins and thus interferes with target binding of the muteins. Alternatively, this additional ligand may compete with binding of the muteins by complexing an epitope distinct from the binding site of the muteins to c-Met by allosteric effects.

### Phage Display

An embodiment of the phage display technique (reviewed in Kay, B. K. et al. (1996), supra; Lowman, H. B. (1997) supra or Rodi, D. J., and Makowski, L. (1999), supra) using temperate M13 phage is given as an example of a selection method. Another embodiment of the phage display technology that may be used for selection of muteins is the hyperphage phage technology as described by Broders et al. (Broders et al. (2003) “Hyperphage. Improving Antibody Presentation in Phage Display” Methods Mol. Biol. 205:295-302). Other temperate phage such as f1 or lytic phage such as T7 may be employed as well. For the exemplary selection method, M13 phagemids are produced which allow the expression of the mutated lipocalin nucleic acid sequence as a fusion protein with a signal sequence at the N-terminus, preferably the OmpA-signal sequence, and with the capsid protein ΔpIII of the phage M13 or fragments thereof capable of being incorporated into the phage capsid at the C-terminus. The C-terminal fragment ΔpIII of the phage capsid protein comprising amino acids 217 to 406 of the wild type sequence is preferably used to produce the fusion proteins. Especially preferred in one embodiment is a C-terminal fragment of ΔpIII, in which the cysteine residue at position 201 is deleted or is replaced by another amino acid.

The fusion protein may comprise additional components such as an affinity tag, which allows the immobilization, detection and/or purification of the fusion protein or its parts. Furthermore, a stop codon may be located between the sequence regions encoding the lipocalin or its muteins and the phage capsid gene or fragments thereof, wherein the stop codon, preferably an amber stop codon, is at least partially translated into an amino acid during translation in a suitable suppressor strain.

### Phagemid Expression

The phagemid vector pTLPC27, now also called pTlc27 described in International patent application PCT/EP2007/057971 may be used for the preparation of a phagemid library encoding hTLc muteins. The nucleic acid molecules coding for hTLc muteins may be inserted into the vector using the two BstXI restriction sites. After ligation, a suitable host strain such as E. coli XL1-Blue is transformed with the resulting nucleic acid mixture to yield a large number of independent clones. Alternatively, another vector may be employed in the preparation of a hyperphagemid library such (e.g., pTLPC59 that is used in the Examples of the present application) may also be used for the preparation of the phagemid library. The vector pTLPC59 is identical to the vector pTLc27 with the exception that the library gene construct for phage display is placed under the control of a lac p/o instead of a tet p/o and is genetically fused to the full length gene III of VCSM13 phage.

The resulting library may be subsequently superinfected in liquid culture with an appropriate M13-helper phage or hyperphage to produce functional phagemids. The recombinant phagemid displays the lipocalin mutein on its surface as a fusion with the coat protein pill or a fragment thereof, while the N-terminal signal sequence of the fusion protein is normally cleaved off. On the other hand, it also bears one or more copies of the native capsid protein pill supplied by the helper phage and is thus capable of infecting a recipient, in general a bacterial strain carrying an F- or F′-plasmid. In case of hyperphage display, the hyperphagemids display the lipocalin muteins on their surface as a fusion with the infective coat protein pill but no native capsid protein. During or after infection with helper phage or hyperphage, gene expression of the fusion protein between the lipocalin mutein and the capsid protein pill may be induced, for example by addition of anhydrotetracycline. The induction conditions are chosen such that a substantial fraction of the phagemids obtained displays at least one lipocalin mutein on their surface. In case of hyperphage display induction conditions result in a population of hyperphagemids carrying between three and five fusion proteins consisting of the lipocalin mutein and the capsid protein pill. Various methods are known for isolating the phagemids, such as precipitation with polyethylene glycol. Isolation typically occurs after an incubation period of 6-8 hours.

### Library Screening

The isolated phagemids may then be subjected to selection by incubation with the desired target (i.e., the extracellular domains of c-Met or portions or fragments thereof), wherein the target is presented in a form allowing at least temporary immobilization of those phagemids which carry muteins with the desired binding activity as fusion proteins in their coat. Among the various embodiments known to the person skilled in the art, the target can, for example, be conjugated with a carrier protein such as serum albumin and be bound via this carrier protein to a protein-binding surface, for example polystyrene. Microtiter plates suitable for ELISA techniques or “immuno-sticks” may be used for such an immobilization of the target. Alternatively, conjugates of the target with other binding groups, such as biotin, may be used. The target may then be immobilized on a surface that selectively binds this group, for example microtiter plates or paramagnetic particles coated with streptavidin, neutravidin or avidin. If the target is fused to an Fc portion of an immunoglobulin, immobilization may also be achieved with surfaces, for example microtiter plates or paramagnetic particles, which are coated with protein A or protein G.

Non-specific phagemid-binding sites present on the surfaces may be saturated with a blocking solution such as those used in ELISA techniques. The phagemids are then typically brought into contact with the target immobilized on the surface in the presence of a physiological buffer. Multiple washings remove unbound phagemids. The phagemid particles remaining on the surface are then eluted. For elution, several methods are possible. For example, the phagemids may be eluted by addition of proteases or in the presence of acids, bases, detergents or chaotropic salts or under moderately denaturing conditions. A preferred method is the elution using buffers of pH 2.2, wherein the eluate is subsequently neutralized. Alternatively, a solution of the free target (i.e., the extracellular domains of c-Met or portions or fragments thereof), may be added to compete with the immobilized target for binding to the phagemids or target-specific phagemids may be eluted by competition with immunoglobulins or natural liganding proteins which specifically bind to the target of interest.

Afterwards, E. coli cells are infected with the eluted phagemids. Alternatively, the nucleic acids may be extracted from the eluted phagemids and used for sequence analysis, amplification or transformation of cells in another manner. Starting from the E. coli clones obtained in this way, fresh phagemids or hyperphagemids are again produced by superinfection with M13 helper phages or hyperphage according to the method described above and the phagemids amplified in this way are once again subjected to a selection on the immobilized target. Multiple selection cycles are often necessary to obtain the phagemids with the muteins in sufficiently enriched form.

The number of selection cycles is preferably chosen such that in the subsequent functional analysis at least 0.1% of the clones studied produce muteins with detectable affinity for the given target. Depending on the size and the complexity of the library employed, 2 to 8 cycles may be required.

The selection may also be carried out by means of other methods. Many corresponding embodiments are known to the person skilled in the art or are described in the literature. Moreover, a combination of methods may be applied. For example, clones selected or at least enriched by phage display may additionally be subjected to colony screening. This procedure has the advantage that individual clones may directly be isolated with respect to the production of a tear lipocalin mutein with detectable binding affinity for c-Met or, for example an extracellar domain of c-Met.

### Host Cells

In addition to the use of E. coli as host organism in the phage display technique or the colony screening method, other bacterial strains, yeast or also insect cells or mammalian cells may be used for this purpose. Further to the selection of a tear lipocalin mutein from a random (naïve) library as described above, evolutive methods including limited mutagenesis may also be applied to optimize a mutein that already possesses some binding activity for the target with respect to affinity or specificity for the target after repeated screening cycles.

### Functional Analysis

For the functional analysis of the selected muteins, an E. coli strain may then be infected with the phagemids obtained from the selection cycles and the corresponding double stranded phagemid DNA is isolated. Starting from this phagemid DNA, or also from the single-stranded DNA extracted from the phagemids, the nucleic acid sequences of the selected muteins may be determined by the methods known in the art and the amino acid sequence may be deduced. The mutated region or the sequence of the entire tear lipocalin mutein may be subcloned on another expression vector and expressed in a suitable host organism. For example, the vector pTlc26 described in International Patent Application PCT/EP2007/057971 may be used for expression in E. coli strains such as TG1. The muteins of tear lipocalin thus produced may be purified by various biochemical methods. The tear lipocalin muteins produced, for example with pTLc26, carry the affinity peptide Strep-tag II (Schmidt et al., supra) at their C-termini and may therefore preferably be purified by streptavidin affinity chromatography.

### Affinity Maturation

Once a mutein with affinity to c-Met or a domain or a fragment thereof has been selected, it is additionally possible to subject such a mutein to another mutagenesis to subsequently select variants of even higher affinity or variants with improved properties such as higher thermostability, improved serum stability, thermodynamic stability, improved solubility, improved monomeric behavior, improved resistance against thermal denaturation, chemical denaturation, proteolysis, or detergents etc. This further mutagenesis, which in case of aiming at higher affinity may be considered as in vitro affinity maturation, may be achieved by site specific mutation based on rational design or a random mutation.

Another possible approach for obtaining a higher affinity or improved properties is the use of error-prone PCR, which results in point mutations over a selected range of sequence positions of the lipocalin mutein. The error-prone PCR may be carried out in accordance with any known protocol such as the one described by Zaccolo et al. (1996) J. Mol. Biol. 255, 589-603. Other methods of random mutagenesis that are suitable for such purposes include random insertion/deletion (RID) mutagenesis as described by Murakami, H et al. (2002) Nat. Biotechnol. 20, 76-81 or non homologous random recombination (NRR) as described by Bittker, J. A. et al. (2002) Nat. Biotechnol. 20, 1024-1029. If desired, affinity maturation may also be carried out according to the procedure described in WO 00/75308 or Schlehuber, S. et al., (2000) J. Mol. Biol. 297, 1105-1120, where muteins of the bilin-binding protein having high affinity to digoxigenin were obtained. A further approach for improving the affinity is to carry out positional saturation mutagenesis. In this approach small nucleic acid libraries may be created in which amino acid exchanges/mutations are only introduced at single positions within any of the four loop segments defined here (cf., Example 21). These libraries are then directly subjected to a selection step (affinity screening) without further rounds of panning. This approach allows the identification of residues that contribute to improved binding of the desired target and allows identification of hot spots that are important for the binding. With such an approach the identification of key residues within the first two segments (sequence positions 24-36 or 56-58) is possible.

Selection may be performed under conditions, which favor complex formation of the target with muteins that show a slow dissociation from the target (i.e., a low k_{off} rate). Alternatively, selection may be performed under conditions, which favor fast formation of the complex between the mutein and the target (i.e., a high kₒₙ rate). As a further illustrative alternative, the screening may be performed under conditions that select for improved thermostability of the muteins (compared to either wild type tear lipocalin or a mutein that already has affinity towards a pre-selected target) or for a pH stability of the mutein.

c-Met hTLc Muteins

In a further aspect, the present invention is directed to an in vivo imaging agent comprising a mutein of hTLc having detectable binding affinity to c-Met or a domain or portion thereof, wherein this mutein to be included into the in vivo imaging agent is obtainable by or obtained by the above-detailed methods. In one embodiment, the mutein of hTLc obtained according to the above methods includes the substitution of at least one or of both of the cysteine residues occurring at each of the sequences positions 61 and 153 by another amino acid and the mutation of at least one amino acid residue at any one of the sequence positions 26-34, 56-58, 80, 83, 104-106, and 108 of the linear polypeptide sequence of mature hTLc (SEQ. ID. NO:36). The positions 24-36 are comprised in the AB loop, the positions 53-66 are comprised in the CD loop, the positions 69-77 are comprised in the EF loop and the positions 103-110 are comprised in the GH loop in the binding site at the open end of the β-barrel structure of tear lipocalin. The definition of these four loops is used herein in accordance with Flower (Flower, D. R. (1996), supra and Flower, D. R. et al. (2000), supra). Usually, such a mutein comprises at least 2, 3, 4, 5, 6, 8, 10, 12, 14, 15, 16, 17 or 18 mutated amino acid residues at the sequence positions 26-34, 56-58, 80, 83, 104-106, and 108 of the linear polypeptide sequence of mature hTLc. In a specific embodiment, the mutein comprises the amino acid substitutions Cys 61→Ala, Phe, Lys, Arg, Thr, Asn, Tyr, Met, Ser, Pro, or Trp and Cys 153→Ser or Ala. Such a substitution has proven useful to prevent the formation of the naturally occurring disulphide bridge linking Cys 61 and Cys 153, and thus to facilitate handling of the mutein.

In still another embodiment, the mutein used in an in vivo imaging agent comprises at least one additional amino acid substitution selected from Arg 111→Pro and Lys 114→Trp. A mutein may further comprise the cysteine at position 101 of the sequence of native mature hTLc substituted by another amino acid. This substitution may, for example, be the mutation Cys 101→Ser or Cys 101→Thr.

In some embodiments, the lipocalin muteins comprise the wild type amino acid sequence outside the mutated amino acid sequence positions. Alternatively, the lipocalin muteins disclosed herein may also contain amino acid mutations outside the sequence positions subjected to mutagenesis as long as those mutations do not interfere with the binding activity and the folding of the mutein. Such mutations may be accomplished on the DNA level using established standard methods (Sambrook, J. et al. (1989) Molecular Cloning: A Laboratory Manual, 2nd Ed., Cold Spring Harbor Laboratory Press, Cold Spring Harbor, N.Y.). Possible alterations of the amino acid sequence are insertions or deletions as well as amino acid substitutions. Such substitutions may be conservative, wherein an amino acid residue is replaced with a chemically similar amino acid residue. Examples of conservative substitutions are the replacements among the members of the following groups: (1) alanine, serine, and threonine; (2) aspartic acid and glutamic acid; (3) asparagine and glutamine; (4) arginine and lysine; (5) isoleucine, leucine, methionine, and valine; and (6) phenylalanine, tyrosine, and tryptophan. One the other hand, it is also possible to introduce non-conservative alterations in the amino acid sequence. In addition, instead of replacing single amino acid residues, it is also possible to either insert or delete one or more continuous amino acids of the primary structure of tear lipocalin as long as these deletions or insertion result in a stable folded/functional mutein (see for example, the experimental section in which muteins with truncated N- and C-terminus are generated).

Such modifications of the amino acid sequence include directed mutagenesis of single amino acid positions to simplify sub-cloning of the mutated lipocalin gene or its parts by incorporating cleavage sites for certain restriction enzymes. In addition, these mutations may also be incorporated to further improve the affinity of a lipocalin mutein for a given target. Furthermore, mutations may be introduced to modulate certain characteristics of the mutein such as to improve folding stability, serum stability, protein resistance or water solubility or to reduce aggregation tendency, if necessary. For example, naturally occurring cysteine residues may be mutated to other amino acids to prevent disulphide bridge formation. However, it is also possible to mutate other amino acid sequence position to cysteine to introduce new reactive groups, for example for the conjugation to other compounds, such as polyethylene glycol (PEG), hydroxyethyl starch (HES), biotin, peptides or proteins, or for the formation of non-naturally occurring disulphide linkages.

Exemplary possibilities of such a mutation to introduce a cysteine residue into the amino acid sequence of a hTLc mutein include the substitutions Thr 40→Cys, Glu 73→Cys, Arg 90→Cys, Asp 95→Cys, Lys 121→Cys, Asn 123→Cys and Glu 131→Cys. The generated thiol moiety at the side of any of the amino acid positions 40, 73, 90, 95, 121, 123 and/or 131 may be used to PEGylate or HESylate the mutein, for example, to increase the serum half-life of a respective tear lipocalin mutein. The mutein S244.2-H08 into which a cysteine is introduced at any these sequence positions (see Example 9) is an illustrative example. The side chain of any of the cysteine residues may be used not only for conjugation of serum half-life increasing compounds but as well for conjugation of any conjugation partner such as an organic molecule, an enzyme label, a toxin, a cystostatic agent, a pharmaceutically suitable radioactive label, a fluorescent label, a chromogenic label, a luminescent label, a hapten, digoxigenin, biotin, a metal complex, a metal, or colloidal gold.

The present invention in vivo imaging agents include a mutein of which the first four N-terminal amino acid residues of the sequence of mature hTLc (His-His-Leu-Leu; positions 1-4 of SEQ. ID. NO:36) and/or the last two C-terminal amino acid residues (Ser-Asp; positions 157-158) of the sequence of mature hTLc have been deleted (cf. also the Examples and the attached Sequence Listings).

### Monomers and Oligomers

A tear lipocalin mutein typically exists as monomeric protein. However, the muteins may spontaneously dimerize or form higher oligomers. Although the use of lipocalin muteins that form stable monomers may be preferred for some applications, for example, because of faster diffusion and better tissue penetration, the use of lipocalin muteins that spontaneously form stable homodimers or multimers may be advantageous in other instances, since such multimers may provide for an increased affinity and/or avidity to a given target. Furthermore, oligomeric forms of the lipocalin mutein may have slower dissociation rates or prolonged serum half-life. Dimerization or multimerization may be achieved by fusing respective oligomerization domains such as jun-fos domains or leucine-zippers to muteins or by the use of Duocalins.

### Binding Characteristics

The lipocalin muteins used in an in vivo imaging agent are able to bind c-Met receptor tyrosine kinase or a domain or fragment thereof with detectable affinity, preferably with a dissociation constant of at least 200 nM. In some embodiments, the lipocalin muteins bind c-Met with a dissociation constant for c-Met of at least 100, 20, 1 nM or even less. The binding affinity of a mutein to the c-Met may be measured by a multitude of methods such as fluorescence titration, competition ELISA or surface plasmon resonance (BIAcore).

The complex formation between the respective mutein and c-Met or a domain or fragment thereof is influenced by many different factors such as the concentrations of the respective binding partners, the presence of competitors, pH and the ionic strength of the buffer system used, and the experimental method used for determination of the dissociation constant (KD) such as fluorescence titration, competition ELISA or surface plasmon resonance or even the mathematical algorithm used to evaluate of the experimental data.

Accordingly, KD values (dissociation constant of the complex formed between the respective mutein and its ligand) given here may vary within a certain experimental range, depending on the method and experimental setup that is used for determining the affinity of a particular lipocalin mutein for a given ligand. Thus, there may be a slight deviation in the measured KD values or a tolerance range depending, for example, on whether the KD value was determined by surface plasmon resonance (Biacore) or by competition ELISA.

Specific c-Met Binding Mutein Sequences

In some embodiments, the in vivo imaging agents include a tear lipocalin mutein comprises with respect to the amino acid sequence of mature hTLc at least 6, 8, 10, 12, 14, 16 or 17 amino acid substitutions with respect to the amino acid sequence of mature hTLc (SEQ. ID. NO: 36), which are selected from the group consisting of Arg 26→Thr; Glu 27→Gln; Phe 28→Met, Asp; Glu 30→Leu; Met 31→Ser; Asn 32→Leu; Leu 33→Tyr; Glu 34→Val; Leu 56→Asn; Ile 57→Gln; Ser 58→Ile, Val; Asp 80→Tyr; Lys 83→Ala; Glu 104→Asp; Leu 105→Thr; His 106→Trp; and Lys 108→Gly.

In a specific embodiment, the in vivo imaging agents include a mutein further comprising at least one amino acid substitution of mature hTLc (SEQ. ID. NO: 36) selected from the group consisting of Thr 37→Ser; Met 39→Ile, Leu; Asn 48→Ser; Lys 52→Thr, Met; Met 55→Leu; Lys 65→Arg, Leu; and Ile 89→Ser, Gln, Thr, His.

In a more specific embodiment, the in vivo imaging agents include a mutein of mature hTLc (SEQ. ID. NO: 36) comprising the amino acid substitutions: Arg 26→Thr; Glu 27→Gln; Glu 30→Leu; Met 31→Ser; Asn 32→Leu; Leu 33→Tyr; Glu 34→Val; Leu 56→Asn; Ile 57→Gln; Asp 80→Tyr; Lys 83→Ala; Glu 104→Asp; Leu 105→Thr; His 106→Trp; and Lys 108→Gly.

In other embodiments, the in vivo imaging agents include a mutein may comprise one of the following sets of amino acid substitutions of mature hTLc (SEQ. ID. NO: 36): (1) Arg 26→Thr; Glu 27→Gln; Phe 28→Met; Glu 30→Leu; Met 31→Ser; Asn 32→Leu; Leu 33→Tyr; Glu 34→Val; Leu 56→Asn; Ile 57→Gln; Ser 58→Ile; Asp 80→Tyr; Lys 83→Ala; Glu 104→Asp; Leu 105→Thr; His 106→Trp; and Lys 108→Gly; (2) Arg 26→Thr; Glu 27→Gln; Phe 28→Asp; Glu 30→Leu; Met 31→Ser; Asn 32→Leu; Leu 33→Tyr; Glu 34→Val; Leu 56→Asn; Ile 57→Gln; Ser 58→Val; Asp 80→Tyr; Lys 83→Ala; Glu 104→Asp; Leu 105→Thr; His 106→Trp; and Lys 108→Gly; and (3) Arg 26→Thr; Glu 27→Gln; Phe 28→Asp; Glu 30→Leu; Met 31→Ser; Asn 32→Leu; Leu 33→Tyr; Glu 34→Val; Leu 56→Asn; Ile 57→Gln; Ser 58→Ile; Asp 80→Tyr; Lys 83→Ala; Glu 104→Asp; Leu 105→Thr; His 106→Trp; and Lys 108→Gly.

The in vivo imaging agents including a hTLc mutein may comprise, consists essentially of or consist of any one of the amino acid sequences with an amino acid sequence set forth in any one of SEQ ID NO:1, SEQ ID NOs: 4-9, SEQ ID NOs: 22-26, SEQ ID NOs: 32-35, SEQ ID NOs: 37-41, or of a functional fragment or variant thereof.

### Immunogenicity

Also included in the scope of the present invention are in vivo imaging agents including muteins that have been altered with respect to their potential immunogenicity. Cytotoxic T-cells recognize peptide antigens on the cell surface of an antigen presenting cell in association with a Class I Major Histocompatibility Complex (MHC) molecule. The ability of the peptides to bind to MHC molecules is allele-specific and correlates with their immunogenicity. To reduce immunogenicity of a given protein, the ability to predict which peptides in a protein have the potential to bind to a given MHC molecule is of great value. Approaches that employ a computational threading approach to identify potential T-cell epitopes have been previously described to predict the binding of a given peptide sequence to MHC Class I molecules (Altuvia et al. (1995) J. Mol. Biol. 249: 244-250).

Such an approach may also be utilized to identify potential T-cell epitopes in the in vivo imaging agents and to select of muteins on the basis of its predicted immunogenicity. It may be furthermore possible to subject peptide regions that have been predicted to contain T-cell epitopes to additional mutagenesis to reduce or eliminate these T-cell epitopes and thereby diminish immunogenicity. The removal of amphipathic epitopes from genetically engineered antibodies has been described (Mateo et al. (2000) Hybridoma 19(6):463-471) and may be adapted to the muteins. Muteins thus obtained may possess a minimized immunogenicity, which is desirable for their use in therapeutic and diagnostic applications, such as those described below.

### Conjugated Muteins

For some applications, it is also useful for the in vivo imaging agents including muteins to be conjugated to partner such as signal generators, biokinetic enhancing factors, or cytostatic agents.

In general, the in vivo imaging agents may include any appropriate chemical substance or enzyme, which directly or indirectly generates a detectable compound or signal in a chemical, physical, optical, or enzymatic reaction. An example for a physical reaction and at the same time optical reaction/marker is the emission of fluorescence upon irradiation. Alkaline phosphatase, horseradish peroxidase and β-galactosidase are examples of enzyme labels (and at the same time optical labels) that catalyze the formation of chromogenic reaction products. In general, all labels commonly used for antibodies (except those exclusively used with the sugar moiety in the Fc part of immunoglobulins) may also be used for conjugation to the muteins of the present invention.

### Targeting Conjugates

In one embodiment, the in vivo imaging agents including muteins may also be coupled to a targeting moiety that targets a specific body region to deliver the muteins to a desired region or area within the body. One example wherein such modification may be desirable is the crossing of the blood-brain-barrier. To cross the blood-brain barrier, the tear lipocalin muteins may be coupled to moieties that facilitate the active transport across this barrier (see Gaillard P J, et al., Diphtheria-toxin receptor-targeted brain drug delivery. International Congress Series. 2005 1277:185-198 or Gaillard P J, et al. “Targeted delivery across the blood-brain barrier” Expert Opin Drug Deliv. 2005 2(2): 299-309. Such moieties are for example available under the trade name 2B-Trans™ (to-BBB technologies BV, Leiden, NL).

Regulating Biokinetic Properties of c-Met Binding Muteins

The mutein component of the in vivo imaging agents including muteins may also be conjugated to a moiety that modifies the serum half-life of the mutein (also International Patent Application PCT/EP2007/057971 or also PCT publication WO 2006/56464 where such conjugation strategies are described with reference to muteins of human neutrophil gelatinase-associated lipocalin with binding affinity for CTLA-4). The moiety that extends the serum half-life may be a polyalkylene glycol molecule, hydroxyethyl starch, fatty acid molecules, such as palmitic acid (Vajo & Duckworth (2000), Pharmacol. Rev. 52, 1-9), an Fc part of an immunoglobulin, a CH3 domain of an immunoglobulin, a CH4 domain of an immunoglobulin, albumin or a fragment thereof, an albumin binding peptide, an albumin binding protein, an IgG-Fc-binding protein, or a transferrin.

The albumin binding protein may be a bacterial albumin binding protein, an antibody, an antibody fragment including domain antibodies (see U.S. Pat. No. 6,696,245, for example), a lipocalin mutein or another protein or protein domain with binding activity for albumin. Accordingly, suitable conjugation partners for extending the half-life of a lipocalin mutein include albumin (Osborn, B. L. et al. (2002) “Pharmacokinetic and pharmacodynamic studies of a human serum albumin-interferon-alpha fusion protein in cynomolgus monkeys” J. Pharmacol. Exp. Ther. 303, 540-548), or an albumin binding protein, for example, a bacterial albumin binding domain, such as the one of streptococcal protein G (Konig, T. et al., (1998) “Use of an albumin-binding domain for the selective immobilization of recombinant capture antibody fragments on ELISA plates” J. Immunol. Methods 218, 73-83). Other examples of albumin binding peptides that may be used as conjugation partner are, for instance as described in US Patent Application 2003/0069395 or Dennis (2002), “Albumin binding as a general strategy for improving the pharmacokinetics of proteins.” J. Biol. Chem. 277, pp. 35035-35043).

In other embodiments, albumin itself or a biological active fragment of albumin (e.g., human serum albumin or bovine serum albumin or rat albumin) may be used as conjugation partner of a lipocalin mutein. The albumin or fragment thereof may be recombinantly produced as described in U.S. Pat. No. 5,728,553 or European patent applications EP 0 330 451 and EP 0 361 991. Recombinant human albumin (Recombumin®) Novozymes Delta Ltd. (Nottingham, UK) may be conjugated or fused to a lipocalin mutein to extend the half-life of the mutein.

If the albumin-binding protein is an antibody fragment it may be a domain antibody. Domain Antibodies (dAbs) are engineered to allow precise control over biophysical properties and in vivo half-life to create the optimal safety and efficacy product profile. Domain Antibodies are for example commercially available from Domantis Ltd. (Cambridge, UK and MA, USA).

Using transferrin as a moiety to extend the serum half-life of the in vivo imaging agents including muteins. Accordingly, the muteins may be genetically fused to the N or C terminus, or both, of non-glycosylated transferrin. Non-glycosylated transferrin has a half-life of 14-17 days, and a transferrin fusion protein will similarly have an extended half-life. The transferrin carrier also provides high bioavailability, biodistribution and circulating stability. This technology is commercially available from BioRexis (BioRexis Pharmaceutical Corporation, Pa., USA). Recombinant human transferrin (DeltaFerrin™) for use as a protein stabilizer/half-life extension partner is also commercially available from Novozymes Delta Ltd. (Nottingham, UK).

If an Fc part of an immunoglobulin is used for the purpose to prolong the serum half-life of the muteins, the SynFusion™ technology, commercially available from Syntonix Pharmaceuticals, Inc (MA, USA), may be used. The use of this Fc-fusion technology allows the creation of longer-acting biopharmaceuticals and may for example consist of two copies of the mutein linked to the Fc region of an antibody to improve pharmacokinetics, solubility, and production efficiency.

Yet another alternative to prolong the half-life of a mutein is to fuse to the N- or C-terminus of a mutein long, unstructured, flexible glycine-rich sequences (for example poly-glycine with about 20 to 80 consecutive glycine residues). This approach disclosed in WO2007/038619, for example, has been termed recombinant PEG (rPEG).

If polyalkylene glycol is used as conjugation partner, the polyalkylene glycol may be substituted, unsubstituted, linear or branched. It may also be an activated polyalkylene derivative. Examples of suitable compounds are polyethylene glycol (PEG) molecules as described in WO 99/64016, in U.S. Pat. No. 6,177,074 or in U.S. Pat. No. 6,403,564 in relation to interferon, or as described for other proteins such as PEG-modified asparaginase, PEG-adenosine deaminase (PEG-ADA) or PEG-superoxide dismutase (for example, Fuertges et al. (1990) “The Clinical Efficacy of Poly(Ethylene Glycol)-Modified Proteins” J. Control. Release 11, 139-148). The molecular weight of such a polymer, preferably polyethylene glycol, may range from about 300 to about 70.000 Dalton, including, for example, polyethylene glycol with a molecular weight of about 10.000, of about 20.000, of about 30.000 or of about 40.000 Daltons. Moreover, as described in U.S. Pat. No. 6,500,930 or 6,620,413, carbohydrate oligomers and polymers such as starch or hydroxyethyl starch (HES) may be conjugated to a mutein to extend serum half-life.

If one of the above moieties is conjugated to the hTLc mutein, conjugation to an amino acid side chain may be advantageous. Suitable amino acid side chains may occur naturally in the amino acid sequence of hTLc or may be introduced by mutagenesis. In case a suitable binding site is introduced via mutagenesis, one possibility is the replacement of an amino acid at the appropriate position by a cysteine residue. In one embodiment, such mutation includes at least one of Thr 40→Cys, Glu 73→Cys, Arg 90→Cys, Asp 95→Cys, Lys 121→Cys, Asn 123→Cys or Glu 131→Cys substitution of SEQ. ID. NO:36. The newly created cysteine residue at any of these positions may be used to conjugate the mutein to moiety prolonging the serum half-life of the mutein, such as PEG or an activated derivative thereof.

In another embodiment, to provide suitable amino acid side chains for conjugating one of the above moieties to the muteins artificial amino acids may be introduced by mutagenesis. Generally, such artificial amino acids are designed to be more reactive and thus to facilitate the conjugation to the desired moiety. One example of such an artificial amino acid that may be introduced via an artificial tRNA is para-acetyl-phenylalanine.

### Fusion Constructs

For several applications of the muteins disclosed herein it may be advantageous to use them in the form of fusion proteins. In some embodiments, the hTLc mutein is fused at its N-terminus or its C-terminus to a protein, a protein domain or a peptide such as a chelator, a signal generator and/or an affinity tag.

The fusion partner may confer desirable characteristics to the in vivo imaging agents including hTLc muteins such as enzymatic activity or binding affinity for other molecules. Examples of suitable fusion partners include alkaline phosphatase, horseradish peroxidase, glutathione-S-transferase, the albumin-binding domain of protein G, protein A, antibody fragments, oligomerization domains, lipocalin muteins of same or different binding specificity (which results in the formation of “Duocalins”, cf. Schlehuber, S., and Skerra, A. (2001), Duocalins, engineered ligand-binding proteins with dual specificity derived from the lipocalin fold. Biol. Chem. 382, 1335-1342), or toxins.

In particular, the mutein of the in vivo imaging agents including lipocalin mutein may be fused with a separate enzyme active site such that both components of the resulting fusion protein together act on a given target.

Affinity tags such as the Strep-tag® or Strep-tag® II (Schmidt, T. G. M. et al. (1996) J. Mol. Biol. 255, 753-766), the myc-tag, the FLAG-tag, the His6-tag or the HA-tag or proteins such as glutathione-S-transferase also allow easy detection and/or purification of recombinant proteins are further examples of preferred fusion partners.

A histidine tag (e.g., one or more histidines) may be useful as an appending moiety for a signal generator. Accordingly, one or more histidine may be appended to either termini of the muteins and held as a precursor to a labeled mutein. Alternatively, a mutein with an appended histidine tag may have a signal generator (e.g., ^{99m}Tc) attached.

Proteins with chromogenic or fluorescent properties such as the green fluorescent protein (GFP) or the yellow fluorescent protein (YFP) are suitable fusion partners for a lipocalin mutein as well.

In some in vivo imaging agents including, the naturally occurring disulfide bond between Cys 61 and Cys 153 is removed from the hTLc mutein. Accordingly, such muteins (or any other tear lipocalin mutein that does not comprise an intramolecular disulfide bond) may be produced in a cell compartment having a reducing redox milieu, for example, in the cytoplasma of Gram-negative bacteria. In case a lipocalin mutein comprises intramolecular disulfide bonds, it may be preferred to direct the nascent polypeptide to a cell compartment having an oxidizing redox milieu using an appropriate signal sequence. Such an oxidizing environment may be provided by the periplasm of Gram-negative bacteria such as E. coli, in the extracellular milieu of Gram-positive bacteria or in the lumen of the endoplasmic reticulum of eukaryotic cells and usually favors the formation of structural disulfide bonds. It is, however, also possible to produce a mutein in the cytosol of a host cell, preferably E. coli. In this case, the polypeptide may either be directly obtained in a soluble and folded state or recovered in form of inclusion bodies, followed by renaturation in vitro. A further option is the use of specific host strains having an oxidizing intracellular milieu, which may thus allow the formation of disulfide bonds in the cytosol (Venturi M, et al. “High level production of functional antibody Fab fragments in an oxidizing bacterial cytoplasm.” J. Mol. Biol. 315, 1-8.).

Chemical Synthesis of c-Met Binding Muteins

A mutein of the in vivo imaging agents may not necessarily be generated or produced only by use of genetic engineering. Rather, a lipocalin mutein may also be obtained by chemical synthesis such as Merrifield solid phase polypeptide synthesis. It is for example possible that promising mutations are identified using molecular modeling and then to synthesize the wanted (designed) polypeptide in vitro and investigate the binding activity for a given target. Methods for the solid phase and/or solution phase synthesis of proteins are known in the art (reviewed, e.g., in Lloyd-Williams, P. et al. (1997) Chemical Approaches to the Synthesis of Peptides and Proteins. CRC Press, Boca Raton, Fields, G. B., and Colowick, S. P. (1997) Solid-Phase Peptide Synthesis. Academic Press, San Diego, or Bruckdorfer, T. et al. (2004) Curr. Pharm. Biotechnol. 5, 29-43).

### Pharmaceutical Compositions & Administration

The invention also relates to a diagnostic compositions comprising at least one in vivo imaging agents including a mutein of hTLc or a fusion protein or conjugate thereof disposed in a pharmaceutically acceptable excipient.

The in vivo imaging agents including lipocalin muteins may be administered to a subject via any parenteral or non-parenteral (enteral) route that is therapeutically effective for proteinaceous drugs. Parenteral application methods comprise, for example, intracutaneous, subcutaneous, intramuscular, intratracheal, intranasal, intravitreal or intravenous injection and infusion techniques, e.g. in the form of injection solutions, infusion solutions or tinctures, as well as aerosol installation and inhalation, e.g. in the form of aerosol mixtures, sprays or powders. An overview about pulmonary drug delivery, i.e. either via inhalation of aerosols (which may also be used in intranasal administration) or intracheal instiallation is given by J. S. Patton et al. The lungs as a portal of entry for systemic drug delivery. Proc. Amer. Thoracic Soc. 2004 Vol. 1 pages 338-344, for example). Non-parenteral delivery modes are, for instance, orally, e.g. in the form of pills, tablets, capsules, solutions or suspensions, or rectally, e.g. in the form of suppositories. The muteins may be administered systemically or topically in formulations containing conventional non-toxic pharmaceutically acceptable excipients or carriers, additives and vehicles as desired.

In one embodiment of the present invention, the agent is administered parenterally to a mammal, and in particular to humans. Corresponding administration methods include, but are not limited to, for example, intracutaneous, subcutaneous, intramuscular, intratracheal or intravenous injection and infusion techniques, e.g. in the form of injection solutions, infusion solutions or tinctures as well as aerosol installation and inhalation, e.g. in the form of aerosol mixtures, sprays or powders. A combination of intravenous and subcutaneous infusion and/or injection might be most convenient in case of compounds with a relatively short serum half-life. The pharmaceutical composition may be an aqueous solution, an oil-in water emulsion or a water-in-oil emulsion.

Accordingly, the in vivo imaging agent including tear lipocalin muteins may be formulated into compositions using pharmaceutically acceptable ingredients as well as established methods of preparation (Gennaro, A. L. and Gennaro, A. R. (2000) Remington: The Science and Practice of Pharmacy, 20th Ed., Lippincott Williams & Wilkins, Philadelphia, Pa.). To prepare the pharmaceutical compositions, pharmaceutically inert inorganic or organic excipients may be used. To prepare e.g. pills, powders, gelatin capsules or suppositories, for example, lactose, talc, stearic acid and its salts, fats, waxes, solid or liquid polyols, natural and hardened oils may be used. Suitable excipients for the production of solutions, suspensions, emulsions, aerosol mixtures or powders for reconstitution into solutions or aerosol mixtures prior to use include water, alcohols, glycerol, polyols, and suitable mixtures thereof as well as vegetable oils.

The composition may also contain additives, such as, for example, fillers, binders, wetting agents, glidants, stabilizers, preservatives, emulsifiers, and furthermore solvents or solubilizers or agents for achieving a depot effect. The latter is that fusion proteins may be incorporated into slow or sustained release or targeted delivery systems, such as liposomes and microcapsules.

The formulations may be sterilized by numerous means, including filtration through a bacteria-retaining filter, or by incorporating sterilizing agents in the form of sterile solid compositions which may be dissolved or dispersed in sterile water or other sterile medium just prior to use.

Diagnostic Methods Using c-Met-binding Muteins

The present disclosure relates to in vivo imaging agents including muteins that are useful to assessing qualitative or quantitative c-Met expression to diagnose or stage a disease condition associated with c-Met expression, such as cancer. In alternative embodiments, qualitative or quantitative c-Met expression may be determined in vivo and used to establish the efficacy of therapies used to treat or ameliorate the symptoms of a disease condition associated with c-Met expression, such as cancer.

Additional sequence may be added to the muteins to impart selected functionality. A signal generator may be incorporated into the polypeptide at a terminal position or at an internal position to create an in vivo imaging agent. Suitable examples of signal generators may include a radionuclide, a paramagnetic ion, a chemiluminescent agent, or a fluorophore. Specific radionuclide useful as in vivo imaging agents include ¹¹C, ¹⁸F, ⁶⁷Ga, ⁶⁸Ga, ^{94m}Tc, ^{99m}Tc, ⁶⁴Cu, ⁶⁷Cu, ¹²³I, or ¹²⁴I. Specific paramagnetic agents for use as in vivo imaging agents include ⁶⁷Ga, ⁶⁸Ga, ⁶⁴Cu, or ⁶⁷Cu. And, specific optical imaging agents useful as in vivo imaging agents include cyanine dyes and quantum dots.

The imaging modality may include positron emission tomography (“PET”), optical, single photon emission computed tomography (“SPECT”), or magnetic resonance imaging (“MRI”). The muteins may be labeled with a signal generator appropriate to the selected imaging modality. Although the signal generator may be incorporated in a variety of fashions with a variety of different radioisotopes, such radiolabeling should be carried out in a manner such that the high binding affinity and specificity of the unlabeled c-Met binding mutein is not significantly affected.

A radionuclide may be attached to the c-Met binding mutein via an imidazole-containing moiety such as a histidine residue. In some embodiments, the histidine residue may be part of a multi-histidine tag (e.g., a tris-histidine tag or a hexa-histidine tag, also referred to as “his6”) that is attached to either termini of the mutein.

In some other embodiments, a radionuclide may be attached to the mutein via a linker such as a bis amine-oxime or a hydrazine nicotinic acid (e.g., cPn216 or HYNIC, respectively). In such embodiments, the linker is bound to a thiol-containing amino acid (e.g., cysteine) residue present in the mutein through a maleimide group on the linker.

In alternative embodiments, the signal generator is attached to the mutein via a chelator (e.g., NOTA, DOTA, or DTPA). In such embodiments, the chelator may be bound to a thiol-containing amino acid residue (e.g., cysteine) present in the mutein.

To assess the target levels, a labeled imaging agent is delivered to a subject. Typically, the subject is a mammal and can be human. The labeled imaging agent is delivered to a subject by a medically appropriate means. Thus, in some embodiments, the in vivo imaging agent is administered parenterally via intracutaneous, subcutaneous, intramuscular, intratracheal, intranasal, intravitreal, or intravenous injection or infusion.

After allowing a clearance time according to the label chosen, the amount of imaging agent bound to target is determined by measuring the emitted signal using an imaging modality. The visual and quantitative analyses of the resulting images provide an accurate assessment of the global and local levels of target in the subject.

The in vivo imaging agents provided here in may be employed to detect or diagnose cell proliferative disorders such cancer as liver cancer, colon cancer, colorectal cancer, hepatocellular carcinoma, papillary renal carcinoma, head and neck squamous cell carcinoma (HNSC), lymph nodes metastases of head and neck, or squamous carcinoma. In other embodiments, the in vivo imaging agents provided herein may be employed to evaluate the metastatic potential of tumors.

The concentration of imaging agent in the composition or solutions may vary as required. The concentration may vary from trace amounts to as much as 5% by body weight of the subject but with vary according to the particular imaging modality used. Typically, agent concentrations are selected primarily based on fluid volumes, and viscosities in accordance with the particular mode of administration selected. Preferably, the agent concentration is between about 0.1 and about 1 nmol, more preferably from about 0.1 nmol to about 0.5 nmol. The imaging agent may be present in several ml of injectable solution, as would be determined based on dose, and easily calculated by one of ordinary skill in the art. For example, if the agent is labeled with ¹⁸F, approximately 105 pmol of ¹⁸F yields 10 mCi of a radiation dose initially. This amount of radioactivity is typical and considered safe in the current medical imaging procedures. A typical composition for intravenous infusion may be made to contain 250 ml of sterile Ringer's solution and up to 100 mg, preferably around 10 mg, of the c-Met imaging agent. The composition containing the imaging agent may be combined with a pharmaceutical composition and may be administered subcutaneously, intramuscularly, or intravenously to patients suffering from, or at risk of, c-Met expression-related conditions such as cancer.

In some embodiments, clearance time can be employed to permit the portions of the imaging agent to travel throughout the subject's body and bind to any available c-Met while also permitting the unbound imaging agent to be cleared from the body or from the brain to thereby decrease noise resulting from non-bound imaging agent. The clearance time will vary depending on the label chosen for use and may range from 1 minute to 24 hours.

The imaging agent may be delivered and the imaging taken to determine the amount of c-Met present in a target tissue site as an indication of disease or pre-disease states. The levels of c-Met may be indicative of pre-disease conditions and therapies toward antagonism or other down-regulation of c-Met or its precursors.

### Therapeutic Efficacy

In another aspect, the present methods may be used to determine the efficacy of therapies used in a mammalian subject. By using multiple images over time, the levels of c-Met may be tracked for changes in amount and location using the in vivo imaging agents provided herein. These methods may aid physicians in determining the amount and frequency of therapy needed by an individual subject.

In one embodiment, the in vivo imaging agent is administered and a baseline image is obtained. The therapy to be evaluated is administered to the subject either before or after a baseline images are obtained. After a pre-determined period of time, a second administration of an imaging agent is given. A second or more images are obtained. By qualitatively and quantitatively comparing the baseline and the second image, the effectiveness of the therapy being evaluated may be determined based on a decrease or increase of the signal intensity of the second image or additional images.

### EXAMPLES

Practice will be still more fully understood from the following examples, which are presented herein for illustration only and should not be construed as limiting the invention in any way. The naming conventions used herein for substitutions refer to the sequence provided in SEQ. ID. NO: 36. Thus, for example, a substitution of Cys for Asn at position 123 of SEQ. ID. NO: 36 is shown in SEQ. ID. NO: 35 at position 119.

### Example 1

### Generation of a library with 1.6×10¹⁰ Independent Tlc Muteins

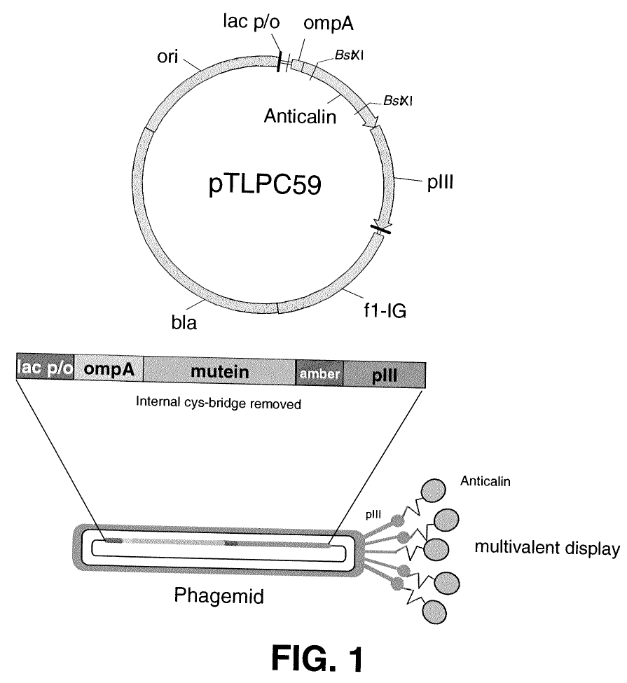

A random library of tear lipocalin (hTLc) with high complexity was prepared essentially as described in Example 1 of PCT application PCT/EP2007/057971 the disclosure of which is incorporated by reference herein with the exception that the library gene construct for phage display pTLPC59 (FIG. 1a and 1b) is placed under the control of a lac p/o instead of a tet p/o and is genetically fused to the full-length gene III of VCSM13 phage.

Tear lipocalin mutein phage production in a multivalent phage display format was realized using M13K07 Hyperphage (Progen) for E. coli infection under standard methods as described in literature (M. Kirsch et al. Journal of Immunological Methods, 301 (2005) pp. 173-185).

### Example 2

### Selection of hTLc Muteins with Affinity for c-Met Receptor

Phagemid display and selection was performed employing the phagemids obtained from Example 1 essentially as described in WO 2005/019256 Example 3 with the following modifications: The target protein (c-Met receptor-Fc, R&D systems) was employed at a concentration of 200 nM and was presented to the library as Fc-fusion protein with subsequent capture of the phage-target complex using protein G beads (Dynal). To select binders that act non-antagonistic to the natural ligand HGF, an additional wash step was introduced using 200 nM of soluble HGF (R&D systems), before c-Met bound library phage were eluted under basic conditions. Four rounds of selection were performed.

### Example 3

### ELISA Identification of c-Met Receptor-Specific Muteins

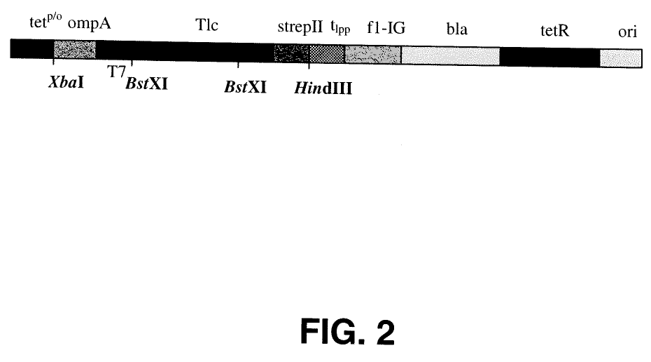

Screening of the muteins selected according to Example 2 was performed essentially as described in Example 3 of WO 2006/56464. Modifications of the protocol are described in the following: Expression vector was pTLPC10 (FIG. 2). Target protein used was c-Met receptor-Fc (R&D Systems) at 1 μg/ml and 3% milk was used as unrelated control target instead of human serum albumin.

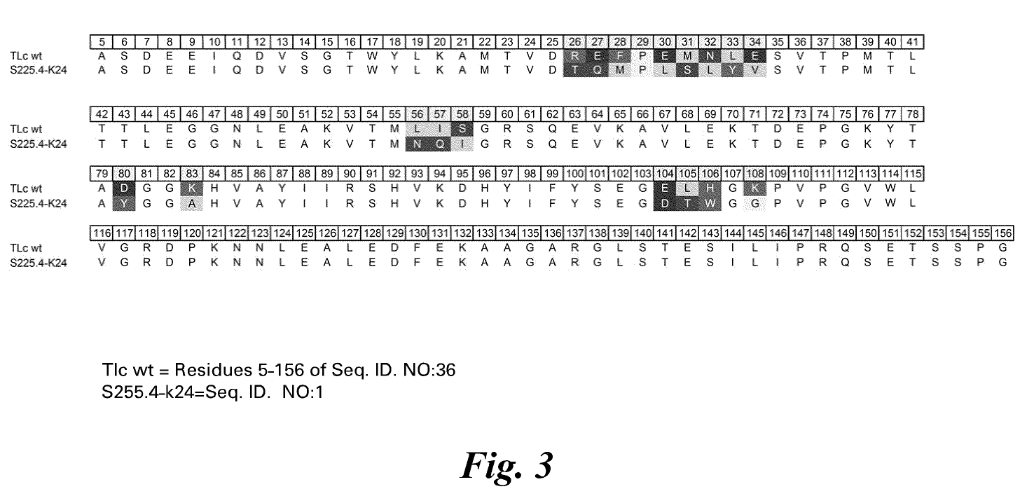

Screening of 2880 clones, selected as described in Example 2, led to the identification of 342 primary hits indicating that successful isolation of muteins from the library had taken place. Using this approach the clone S225.4-K24 (SEQ ID NO: 1) was identified. The sequence of S225.4-K24 is also depicted in FIG. 3.

### Example 4

### Affinity Maturation of S225.4-K24 Using Error-Prone PCR

Generation of a library of variants based on the mutein S225.4-K24 (SEQ ID NO: 1) was performed essentially as described in Example 5 of WO 2006/56464 using the oligonucleotides TL50 bio: TATCTGAAGGCCATGACGGTGGAC (SEQ ID NO:2) and TL51 bio: TGCCCACGAGCCACACCCCTGGGA (SEQ ID NO:3) resulting in a library with 5 substitutions per structural gene on average.

Phagemid selection was carried out as described in Example 2 but employing limited target concentration (2 nM, 0.5 nM and 0.1 nM) of c-Met receptor-Fc, and capturing of target and phagemid complex via anti-human IgG-Fc specific mAb immobilized on a polystyrol plate. Additional selections under identical conditions but with combined target limitation (1 nM) and short incubation time (5 minutes) or target limitation (5 nM, 0.5 nM and 0.1 nM) combined with incubation of phagemids at pH 3, 60° C. for 15 min or pH 10, RT, for 30 min were carried out. Four rounds of selection were performed.

### Example 5

### Affinity Screening of c-Met Receptor-Binding Muteins Using High-Throughput ELISA Screening

Screening was performed as described in Example 3 with the modification that concentrations of 2.5 μg/ml or 0.6 μg/ml of c-Met receptor-Fc (R&D Systems) were used. In total 2880 clones were screened resulting in 1510 hits indicating that successful enrichment of matured muteins from the library had taken place. Additionally in an alternative screening setup monoclonal anti-T7 antibody was coated on a polystyrol plate and expressed muteins were captured via T7-tag prior to incubation with limited concentrations of c-Met receptor-Fc (60 nM, 15 nM and 2.5 nM). Binding of c-Met receptor-Fc was detected using a HRP-conjugated polyclonal antibody against the human IgG-Fc domain.

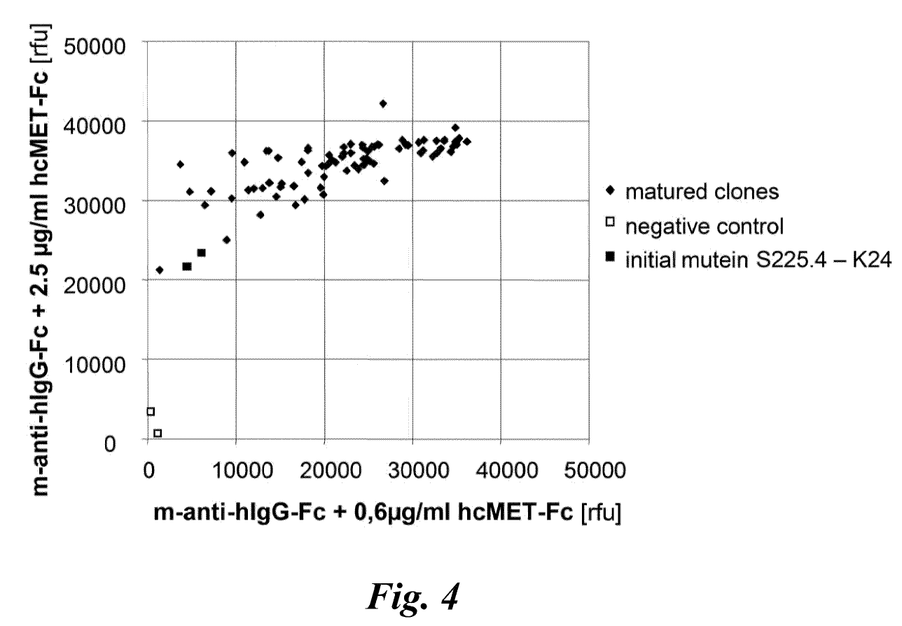

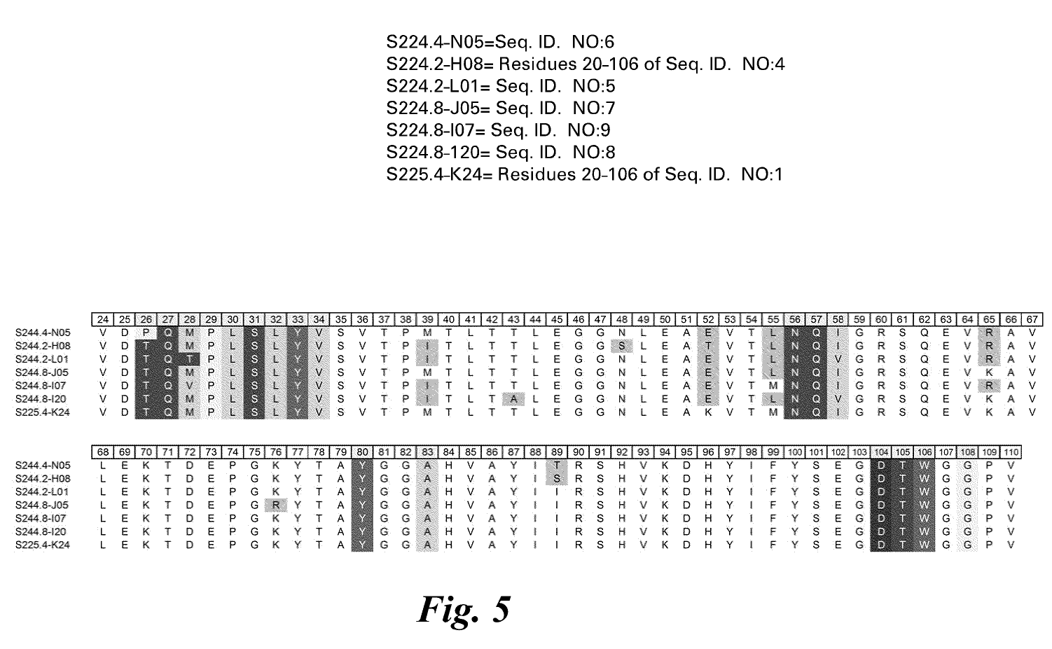

A result from such a screen is depicted in FIG. 4. A large number of muteins selected as described in Example s4 and 5 were identified having improved affinity for c-Met receptor as compared to the mutein S225.4-K24 (SEQ ID NO: 1) that served as the basis for affinity maturation. Using this approach the muteins S244.2-H08 (SEQ. ID. NO: 4), S244.2-L01 (SEQ. ID. NO:5), S244.4-N05 (SEQ. ID. NO:6), S244.5-J05 (SEQ. ID. NO:7), S244.8-120 (SEQ. ID. NO:8), S244.8-107 (SEQ ID NO:9) were identified. These sequences are also depicted in FIG. 5.

### Example 6

### Production of c-Met Receptor-Binding Muteins

For preparative production of c-Met receptor-specific muteins, E. coli K12 strain JM83 harboring the respective mutein encoded on the expression vector pTLPC10 (FIG. 2) was grown in a 2 L shake flask culture in LB-Ampicillin medium according to the protocol described in Schlehuber, S. et al. (J. Mol. Biol. (2000), 297, 1105-1120). When larger amounts of protein were needed, the E. coli strain W3110 harboring the respective expression vector was used for the periplasmatic production via bench top fermenter cultivation in a 1-L or 10-L vessel based on the protocol described in Schiweck, W., and Skerra, A. Proteins (1995) 23, 561-565).

The muteins were purified from the periplasmic fraction in a single step via streptavidin affinity chromatography using a column of appropriate bed volume according to the procedure described by Skerra, A. et al. (2000) “Use of the Strep-tag and streptavidin for detection and purification of recombinant proteins” Methods Enzymol. 326A, 271-304). To achieve higher purity and to remove any aggregated recombinant protein, a gel filtration of the muteins was finally carried out on a Superdex 75 HR 10/30 column (24-ml bed volume, Amersham Pharmacia Biotech) in the presence of PBS buffer. The monomeric protein fractions were pooled, checked for purity by SDS-PAGE, and used for further biochemical characterization.

### Example 7

### Affinity Measurement Using SPR

Affinity measurements were performed essentially as described in Example 9 of WO 2006/56464 with the modifications that approximately 9000 RU of c-Met receptor-Fc (R&D Systems) was directly immobilized on the surface of a CM5 chip (instead of 2000 RU of human CTLA-4 or murine CTLA-4-Fc used as target in WO 2006/56464) and 80 μl of mutein was injected at a concentration of 0.2-0.5 μM (instead of a 40 μl sample purified lipocalin muteins at concentrations of 5 μM-0.3 μM as used in WO 2006/56464). The chip surface was regenerated between measurements by injection of 5-10 μl of 50 mM NaOH pH 10, 2.5 M NaCl. The flow rate was held constant at 10 μl/min.

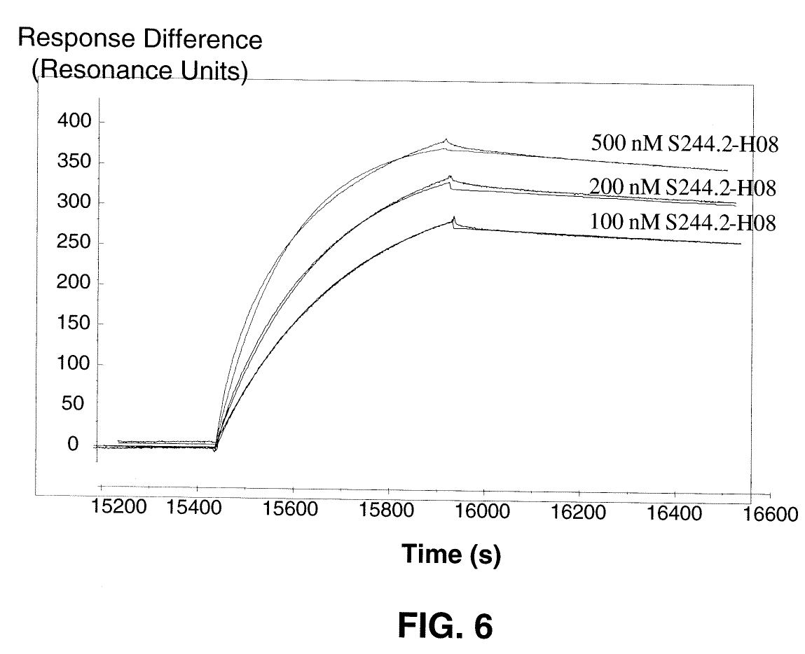

Results from the affinity measurements employing S244.2-H08, S244.2-L01, S244.5-J05, S244.8-120, and are summarized in Table I and evaluation of sensorgrams exemplary for S244.2-H08 is depicted in FIG. 6.

TABLE I

| Mutein | kₒₙ (1/Ms × 10⁴) | K_{off} (1/s 10⁻⁴) | KD [nM] |
|---|---|---|---|
| S244.2-H08 | 1.51 | 1.5 | 9.9 |
| (SEQ. ID. NO: 4) | | | |
| S244.2-L01 | 1.24 | 1.96 | 15.8 |
| (SEQ. ID. NO: 5) | | | |
| S244.4-N05 | 1.1 | 2.64 | 24 |
| (SEQ. ID. NO: 6) | | | |
| S244.5-J05 | 0.9 | 2.09 | 23 |
| (SEQ. ID. NO: 7) | | | |
| S244.8-I20 | 0.87 | 4.1 | 47 |
| (SEQ. ID. NO: 8) | | | |
| S244.8-I07 | 0.93 | 1.45 | 15.5 |
| (SEQ. ID. NO: 9) | | | |

Table I shows affinities of selected muteins for c-Met receptor as determined by surface plasmon resonance (SPR). Mean values were calculated from at least 3 independent measurements.

### Example 8

### Affinity Ranking of Lipocalin Muteins on Intact Cells by Flow Cytometry

Lipocalin muteins were titrated on HT29 cells (ATCCwhich show endogenous expression of HGFR/c-Met. Muteins were tested in 24 1:2 dilutions starting from a 10-μM concentration in a total volume of 30 μl. For each binding reaction, 100,000 cells were incubated in PBS containing 2% Fetal Calf Serum (FCS) for 2 h on at 4° C. Cells were washed twice with PBS, 2% FCS and incubated with 375 ng biotinylated, affinity-purified goat anti-tear lipocalin antiserum per reaction for 30 min. After washing, detection was achieved after further 30 min incubation with Streptavidin-Phycoerythrin. Cells were washed and fluorescence was analyzed on a FACS Calibur flow cytometer. Mean Fluorescence Intensity (MFI) was plotted against mutein concentration and fitted to a sigmoidal dose response curve and EC₅₀ values were determined using GraphPad Prism software.

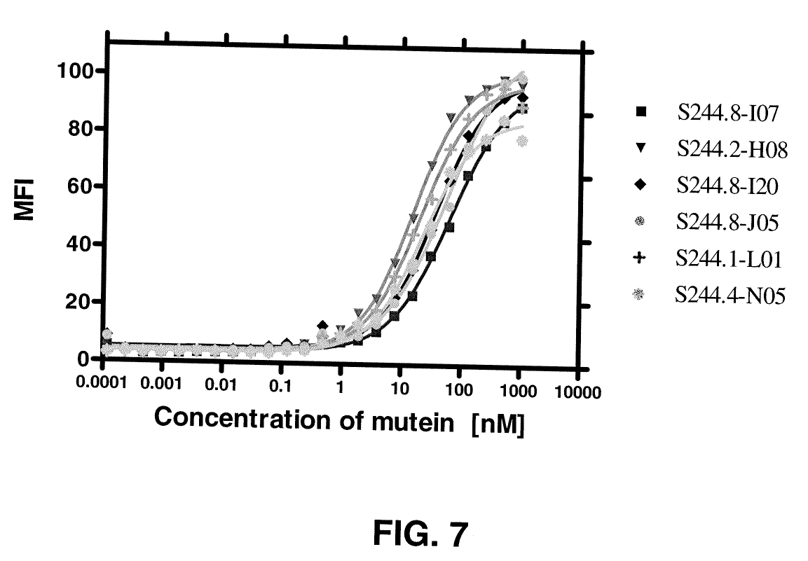

Titration curves from which EC50 values for S244.2-H08 (SEQ. ID. NO:4), S244.2-L01 (SEQ. ID. NO: 5), S244.4-N05 (SEQ. ID. NO:6), S244.5-J05 (SEQ. ID. NO:7), S244.8-120 (SEQ. ID. NO:8), and S244.8-107 (SEQ. ID. NO:9) were determined are depicted in FIG. 7 and calculated EC₅₀ values are summarized in Table II.

TABLE II

| Clone | EC₅₀ [nM] | STD |
|---|---|---|
| S244.2-H08 (SEQ. ID. NO: 4) | 13.2 | 1.3 |
| S244.2-L01 (SEQ. ID. NO: 5) | 16.8 | 1.7 |
| S244.4-N05 (SEQ. ID. NO: 6) | 18.2 | 18.2 |
| S244.8-I07 (SEQ. ID. NO: 9) | 44.8 | 5.9 |
| S244.8-I20 (SEQ. ID. NO: 8) | 28.1 | 4 |
| S244.8-J05 (SEQ. ID. NO: 7) | 37.8 | 5.5 |

Table II shows EC₅₀ values and standard deviations of selected muteins for c-Met receptor as determined by FACS titration on HT29 cells.

### Example 9

### Screening of Lipocalin Mutein-Cys Variants

To provide a reactive group for site-directed coupling with e.g. activated PEG or a pharmaceutically relevant label, an unpaired cysteine residue was introduced by site-directed mutagenesis. The recombinant mutein carrying the free Cys residue was subsequently produced in E. coli as described in Example 6, the expression yield determined and the affinity measured by SPR essentially as described in Example 7.

Cysteine was introduced either instead of the amino acids Thr 40, Asp 95, Arg 90, Lys 121, Asn 123 or Val 93 employing pair-wise the oligonucleotides are shown in Table III below.

TABLE III

| | | |
|---|---|---|
| SEQ ID NO: 10 | H08_T40C | CGTCTCGGTAACACCCATATGCCTCACG |
| | forward | ACCCTGGAAGGG |
| SEQ ID NO: 11 | H08_T40C | CCCTTCCAGGGTCGTGAGGCATATGGGT |
| | reverse | GTTACCGAGACG |
| SEQ ID NO: 12 | H08_D95C | CAGGTCGCACGTGAAGTGCCACTACATC |
| | forward | TTTTACTCTGAGGG |
| SEQ ID NO: 13 | H08_D95C | CCCTCAGAGTAAAAGATGTAGTGGCACTT |
| | reverse | CACGTGCGACCTG |
| SEQ ID NO: 14 | H08_R90C | CGTGGCATACATCAGCTGCTCGCACGTG |
| | forward | AAGGATCAC |
| SEQ ID NO: 15 | H08_R90C | GTGATCCTTCACGTGCGAGCAGCTGATG |
| | | TATGCCACG |
| SEQ ID NO: 16 | A22_ | GGCAGAGACCCCTGCAACAACCTGGAAG |
| | K121C | CCTTG |
| | forward | |
| SEQ ID NO: 17 | A22_ | CAAGGCTTCCAGGTTGTTGCAGGGGTCT |
| | K121C | CTGCC |
| | reverse | |
| SEQ ID NO: 18 | A22_ | GGCAGAGACCCCAAGAACTGCCTGGAAG |
| | N123C | CCTTGGAG |
| | forward | |
| SEQ ID NO: 19 | A22_ | GGCAGAGACCCCAAGAACTGCCTGGAAG |
| | N123C | CCTTGGAG |
| | reverse | |
| SEQ ID NO: 20 | H08_V93C | CATCAGCAGGTCGCACTGCAAGGATCAC |
| | forward | TACATCTTTTAC |
| SEQ ID NO: 21 | H08_V93C | GTAAAAGATGTAGTGATCCTTGCAGTGC |
| | reverse | GACCTGCTGATG |

Exemplary, results from the Cys-screening of the c-Met receptor-specific mutein S244.2-H08 (SEQ ID NO: 4) are given in Table IV below.

TABLE IV

| Mutein | Yield [μg/L] | Affinity [nM] |
|---|---|---|
| S244.2-H08_K121C | 31 | 8 |
| S244.2-H08_N123C | 51 | 14 |
| S244.2-H08_D95C | 15 | 11 |
| S244.2-H08_R90C | 55 | 35 |
| S244.2-H08_T40C | 63 | 40 |
| S244.2-H08_V93C | 31 | 21 |
| S244.2-H08 | 200 | 8 |

SPR-affinities for c-Met receptor of the mutein S244.2-H08 and mutants thereof comprising amino acid exchanges Thr 40→Cys (SEQ ID NO: 22), Asn 123→Cys (SEQ ID NO: 23), Asp 95→Cys (SEQ ID NO: 24), Arg 90→Cys (SEQ ID NO: 25), and Lys 121→Cys (SEQ ID NO: 26).

### Example 10

### Affinity Maturation of the Mutein S225.4-K24 Using a Site-Directed Random Approach

A library of variants based on the mutein S225.4-K24 (SEQ ID NO: 1) was designed by randomization of the residue positions 28, 39, 52, 5, 58, 65, and 89 to allow for all 20 amino acids on these positions. The library was constructed essentially as described in Example 1 with the modification that three randomized PCR fragments were generated employing pair wise the deoxynucleotides K24_{—}1 (SEQ. ID. NO:27) (covering position 28) and K24_{—}2 (SEQ. ID. NO:28) (covering position 39), K24_{—}3, (SEQ. ID. NO:29) (covering positions 52, 55, and 58) and K24_{—}4: (SEQ. ID. NO:30) (covering position 65), K24_{—}5: (SEQ. ID. NO:31) (covering position 89) and TL51bio (SEQ. ID. NO:3) instead of TL46, TL47, TL48 and TL49, respectively. Phagemid display and selection was performed employing the phagemids essentially as described in Example 2 with the following modifications: The target protein was monomeric c-Met receptor without Fc-portion (R&D systems) in a biotinylated form that allows capturing of target:phagemid complex via neutravidin (Pierce) immobilized on a polystyrol plate. Selection was performed using either limited target concentration (1.5 nM and 0.5 nM and 0.1 nM of biotinylated c-Met receptor) or limited target concentration (3 μg/ml, 1 μg/ml, and 0.3 μg/ml) was combined with shorter incubation time (10 min) or a competitive approach using high excess (10 μM) of purified c-Met specific mutein S244.2-H08 (SEQ ID NO.: 4) derived from error prone maturation as described in Example 5. Three rounds of selection were performed.

### Example 11

### Affinity Screening of c-Met Receptor-Binding Muteins Using High-Throughput ELISA Screening

Screening was essentially performed as described in Example 5 in alternative screening setups with the following modifications: i) monoclonal anti-T7 antibody was coated on a polystyrol plate and expressed muteins were captured via T7-tag prior to incubation with limited concentrations of monomeric c-Met receptor-bio (50 nM, 10 nM and 2.5 nM). Binding of target was detected using a HRP (horseradish peroxidase)-conjugated Extravidin; ii) Biotinylated c-Met receptor (1 μg/ml) was captured on neutravidin plates; binding of expressed c-Met specific muteins was detected via HRP-conjugated anti-T7 mAb (Novagen) either after unlimited (60 min) or limited (5 min) incubation time; and iii) the extract containing the c-Met-binding muteins was heated to 70° C. for 1 hour; and iv) Biotinylated c-Met receptor (R&D Systems, 2.5 μg/ml) was captured on neutravidin plates. Mutein extracts were preincubated with high excess (1 μM) of purified c-Met specific mutein S244.2-H08 (SEQ. ID. NO:4) from Example 5 as a competitor for target binding. Binding of expressed c-Met specific muteins was detected via HRP-conjugated anti-T7 mAb (Novagen).

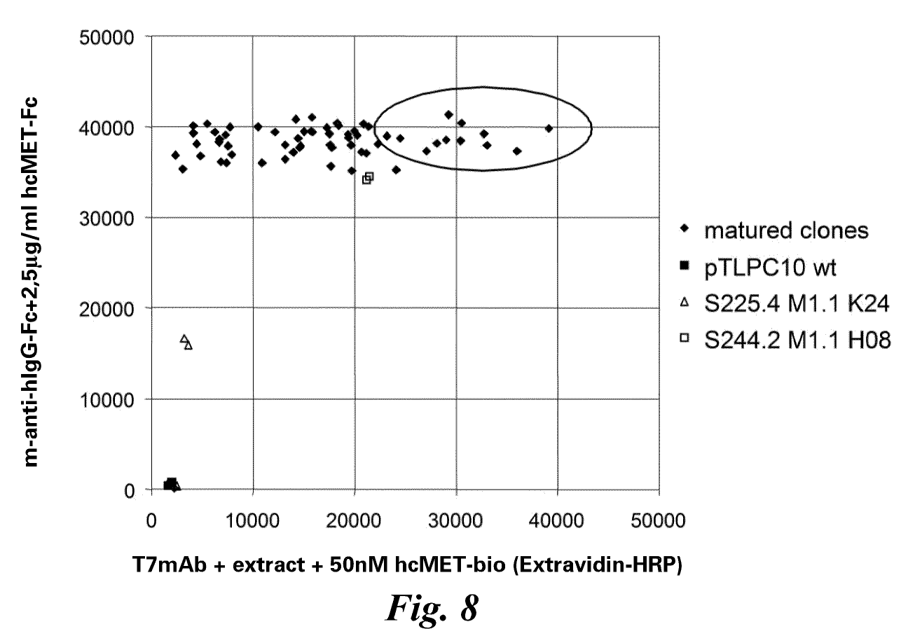

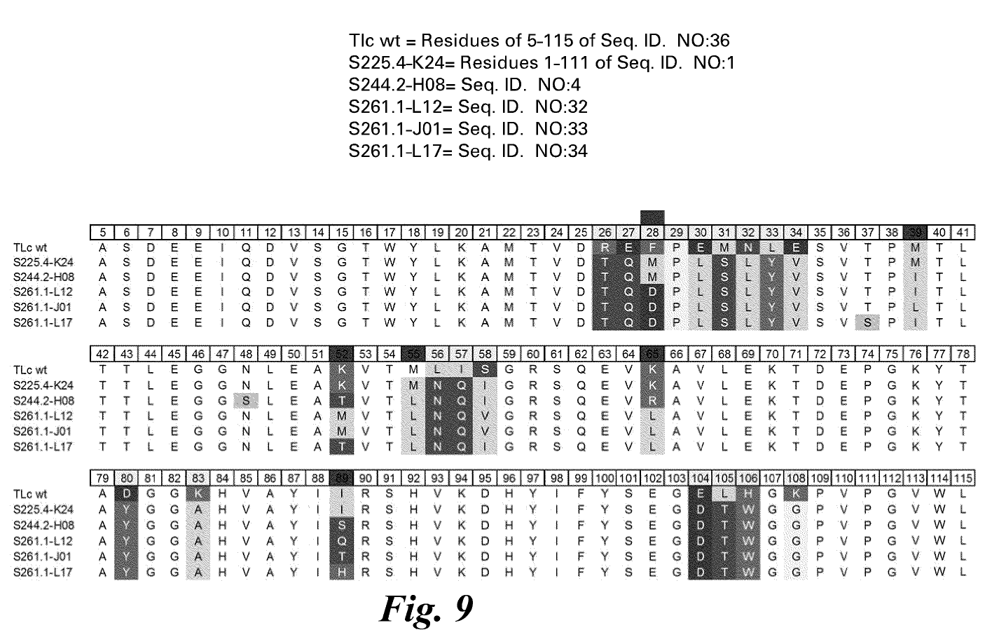

A result from such a screen is depicted in FIG. 8. A large number of muteins selected as described in Example 12 and 13 were identified having improved affinity for c-Met receptor as compared to the mutein S225.4-K24 (SEQ ID NO.:1), which served as the basis for affinity maturation. Using this approach the muteins S261.1-L12, S261.1-J01, S261.1-L17 (SEQ ID NOs.:32-34) were identified. The sequences of S261.1-L12, S261.1-J01, S261.1-L17 are also depicted in FIG. 9 together with the sequence of S225.4-K24 (SEQ. ID. NO:1) and S244.2-H08 (SEQ. ID. NO:4), which is a mutein derived from error-prone maturation as described in Example 5.

### Example 12

### Production of c-Met Receptor-Binding Muteins in a His-Tagged Format

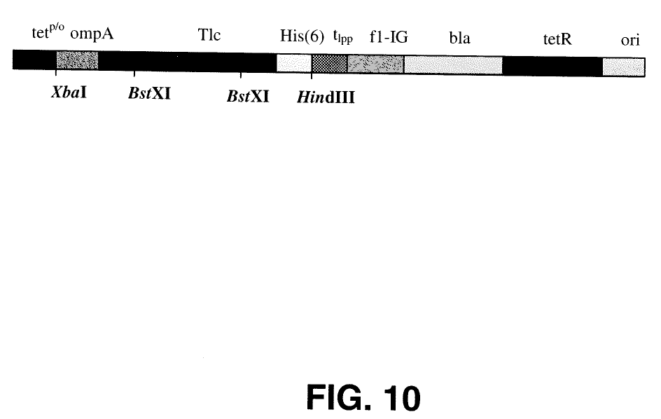

Periplasmatic production via fermenter cultivation in a 0.75 L bioreactor was essentially performed according to Example 6 with the modification that the respective mutein is encoded on expression vector pTLPC47 (FIG. 10) instead of pTLPC10. Vector elements of pTLPC47 are identical to pTLPC10 with the modification that pTLPC47 codes for an hTLc mutein which is C-terminally fused to a Hexa-His tag and the N-terminally fused T7-tag is removed.

The mutein was purified from the periplasmic fraction in a single step chromatographic protocol with Ni-NTA sepharose (GE) using a column of appropriate bed volume and suitable equipment according to the manufacturers' recommendations.

To achieve higher purity and to remove any aggregated recombinant protein, a gel filtration the muteins was finally carried out on a Superdex 75 HR 10/30 column (24-ml bed volume, Amersham Pharmacia Biotech) in the presence of PBS buffer. The monomeric protein fractions were pooled, checked for purity by SDS-PAGE, and used for further biochemical characterization.

### Example 13

### Affinity Measurement Using SPR

Affinity measurements were performed essentially as described in Example 7. Results from the affinity measurements employing S261.1-L 2, S261.1-J01, S261.1-L17 (SEQ ID NOs.:32-34) and S244.2-H08 (SEQ NO: 4) which is a mutein derived from error prone maturation described in Example 4 and 5 are summarized in Table V.

TABLE V

| | Kₒₙ | | |
|---|---|---|---|
| Mutein | [10⁴ M−¹ s−¹] | K_{off} [10⁻⁴ s⁻¹] | KD [nM] |
| S261.1-L17 | 538 | 1.39 | 2.6 |
| S261.1-L12 | 2.7 | 0.66 | 2.4 |
| S261.1-J01 | 2.86 | 0.65 | 2.3 |
| S244.2-H08 | 1.8 | 2.58 | 14 |

Affinity improvement of selected muteins from second affinity maturation as described in Examples 10 and 11 compared to mutein S244.2-H08 (SEQ. ID. NO:4) from first affinity maturation cycle determined by SPR.

### Example 14

### Affinity Ranking of Tear Lipocalin Muteins on Intact Cells by Flow Cytometry

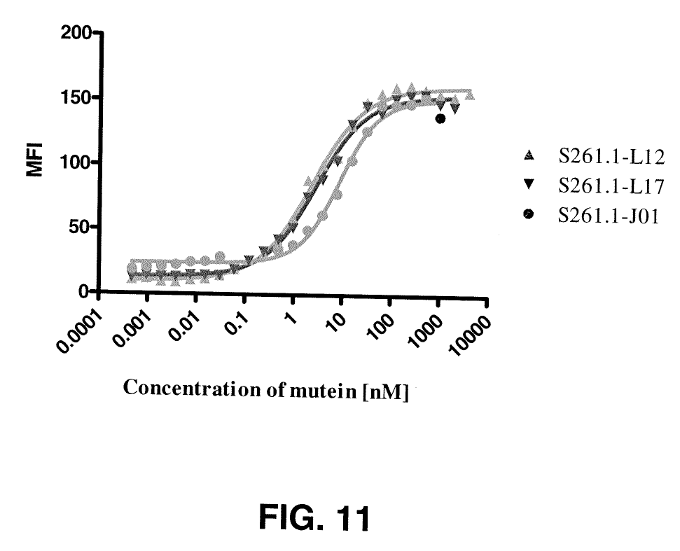

Tear lipocalin muteins were titrated on HT29 cells (ATCC) essentially as described in Example 8. Titration curves from which EC50 values for S261.1-L12, S261.1-J01, S261.1-L17 (SEQ ID NOs.:32-34) were determined are depicted in FIG. 11 and calculated EC50 values are summarized in Table VI.

TABLE VI

| Mutein | EC₅₀ [nM] |
|---|---|
| S261.1-L12 | 2.3 |
| S261.1-J01 | 8.5 |
| S261.1-L17 | 2.8 |

Table V shows EC₅₀ values of selected muteins for c-Met receptor as determined by FACS titration on HT29 cells.

### Example 15

### Identification of Non-Antagonistic Binding Mode of c-Met Receptor-Specific Mutein Using an HGF Competition ELISA

The mode of the interaction between HGF (Hepatocyte-growth factor, R&D Systems) and c-Met receptor by the selected c-Met specific muteins was evaluated in a competition ELISA. Therefore, a constant concentration of 2.5 μg/ml c-Met receptor-Fc (R&D Systems) was captured via anti-human IgG-Fc specific mAb (Jackson Immuno Research) which was immobilized on the surface of a polystyrol plate before. In the following the target was incubated for 1 hour at room temperature with a dilution series of c-Met-specific mutein starting from 100 nM in a two-step dilution series and binding takes place either in absence or presence of 300 nM HGF as competitor. Bound c-Met receptor specific mutein was detected using polyclonal biotinylated anti-lipocalin 1 antibody (R&D Systems) and bound HGF was detected using polyclonal anti-HGF-bio antibody (R&D Systems). In both cases HRP-conjugated Extravidin (Sigma) was employed as secondary detection reagent.

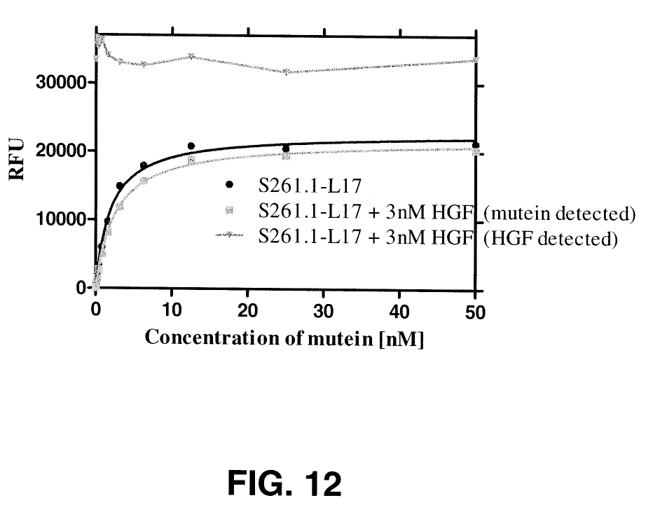

Result from measurement employing the mutein S261.1-L7 (SEQ NO.:34) serve as an example and is depicted in FIG. 12. KD values determined from mutein titration curves are summarized in Table VII.

TABLE VII

| Clone | KD (nM) − HGF | KD (nM) + HGF |
|---|---|---|
| S261.1-L17 (SEQ. ID. NO: 34) | 1.9 | 2.5 |

Table VI shows Non-antagonistic ability and affinities for c-Met receptor of selected tear lipocalin mutein S261.1-L17 (SEQ. ID. NO: 34) as determined by competition ELISA.

### Example 16

### Determination of Thermal Denaturation for c-Met-Binding Muteins by Use of CD Spectroscopy

Circular dichroism measurements were performed essentially as described in Example 14 of the International patent application WO2006/056464, with the modification that the wavelength used was 230 nM and the mutein concentration was 250 μg/ml. The melting temperatures Tm of the tear lipocalin muteins S261.1-L12, S261.1-J01, S261.1-L17 (SEQ ID NOs.:32-34, respectively) and H08 (SEQ ID NO.: 4) are summarized in Table VIII.

TABLE VIII

| Mutein | Tm [° C.] |
|---|---|
| S261.1-L12 | 63.2 |
| S261.1-J01 | 59 |
| S261.1-L17 | 64.7 |
| S244.2-H08 | 65.5 |

### Example 17

### Production of c-Met-Specific Mutein S261.1-L12_C123 with Unpaired Cysteine at Position 123

Preparative production of c-Met-specific mutein S261.1-L12_C123 (SEQ NO:35) was performed essentially as described in Example 6 with the modification that amino acid Asn 123 was changed to cysteine to introduce an unpaired cysteine for subsequent site-directed conjugations. Cysteine 123 was selected according to transfer the results from cysteine-screen described in Example 9, which demonstrates good expression yield and affinity compared to the original mutein S261.1-L12 (SEQ NO:32) without the unpaired cysteine.

### Example 18

### Site-Directed Conjugation of HYNIC to S261.1-L12_C123

Purified mutein S262.1-I12_C123 (SEQ. ID. NO:35) from Example 17 was used at a concentration of 0.8 mg/ml in PBS buffer pH 7.4 and unpaired cysteine was activated by addition of 100 mM TCEP (Sigma) to a final concentration of 1 mM. After 2 hours incubation at room temperature unreacted TCEP excess was removed by gel filtration employing a NAP-5 column (GE) according to manufacturers' recommendations. A 10-molar excess of HYNIC (3-N-maleimido-6-hydraziniumpyridine hydrochloride purchased from SoluLink) was added and incubated for 2 h at room temperature. To remove the unreacted HYNIC from the conjugated mutein the reaction mixture was concentrated in an Ultracentricon (Amicon) and washed at least for 5 times using appropriate volumes of PBS buffer.

### Example 19

### Affinity Measurements of HYNIC-Conjugated c-Met-Specific Mutein S261.1-L12_C123 on Intact Cells by Flow Cytometry

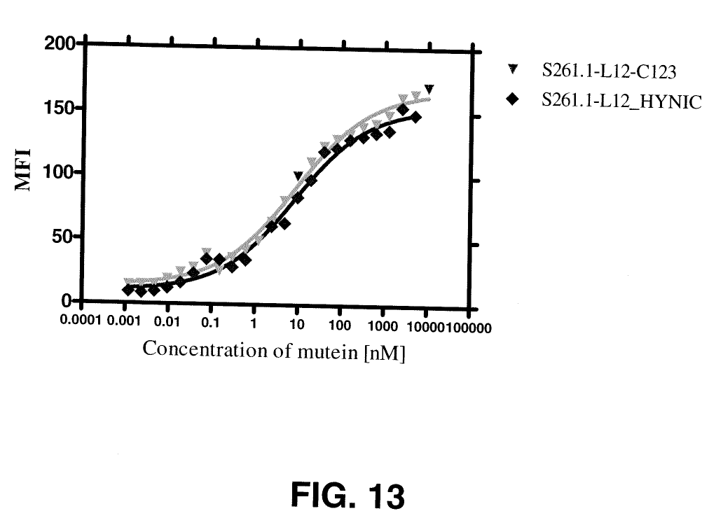

c-Met-specific mutein S261.1-L12_C123 (SEQ. ID. NO:35) with and without conjugated HYNIC was titrated on HT29 cells (ATCC) as described in Example 8. Titration curves from which EC50 values were determined are depicted in FIG. 13 and calculated EC50 values are summarized in Table IX as determined by FACS titration on HT29 cells.

TABLE IX

| Mutein | EC50 [nM] |
|---|---|
| S261.1-L12_C123 | 8.6 |
| S261.1-L12_C123_HYNIC | 8.3 |

### Example 20

### pH Stability of c-Met-Specific Muteins

Purified mutein S261.1-J01 from Example 12 was incubated for 60 min at different pH ranging between pH 3 and pH 9.2. After neutralization to pH 7.4 the mutein was analyzed via size-exclusion chromatography by employing an analytical Superdex 75 column (GE Healthcare) according to manufacturer's recommendations.

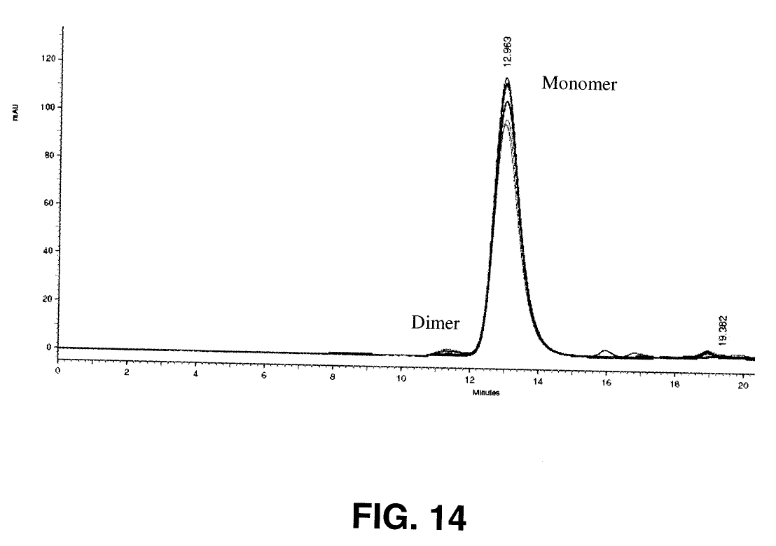

No alteration of the mutein could be detected during the incubation period as judged by HPLC-SEC, except for pH 5-6 which is the range around the pl of the mutein some degree of dimerization occurred as depicted in FIG. 14.

### Example 21

### Positional Saturation Mutagenesis

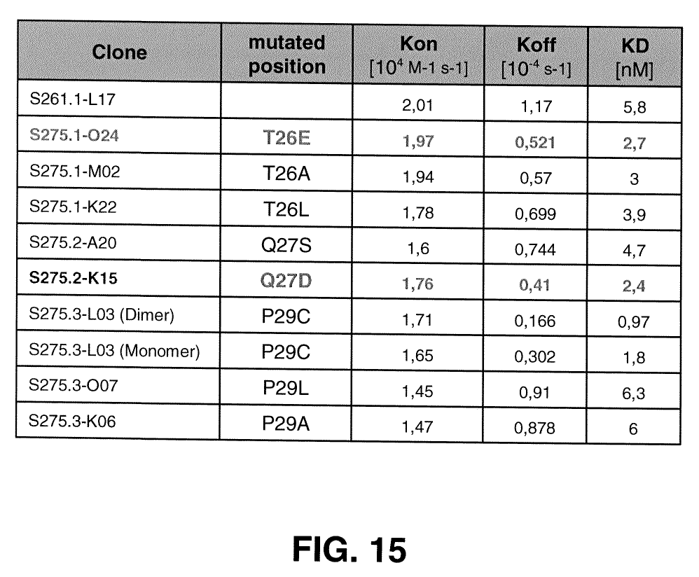

Site-specific mutagenesis was carried out at sequence positions 26, 27 and 29 of the affinity-maturated tear lipocalin muteins S2261.1-L17 (SEQ. ID. NO:34), O24, M02, K22, A22, K15, L03, O07 and K06 to assess whether the binding affinity may be significantly influenced. As shown in FIG. 15, all created mutants showed similar affinities.

### Example 22

### c-Met Binding by Unlabeled His6-Tagged Muteins hTLc (SEQ ID NO:36), S244.2-L01 (SEQ. ID. NO:37), and S244.2-H08 (SEQ. ID. NO:38)

Affinity measurements. Binding interactions between the muteins against the c-Met antigen were measured in vitro using surface plasmon resonance (SPR) detection on a Biacore 3000 instrument (GE Healthcare, Piscataway, N.J.). The extracellular domain of the c-Met antigen was obtained as a c-Met extracellular domain-fc chimera from R&D Systems (Minneapolis, Minn.) and covalently attached to a CM-5 dextran-functionalized sensor chip (GE Healthcare, Piscataway, N.J.) pre-equilibrated with HBS-EP buffer (0.01 M HEPES pH 7.4, 0.15 M NaCl, 3 mM EDTA, 0.005% v/v surfactant P20) at 10 μL/min and subsequently activated with EDC and NHS. The Fc-c-Met (20 μg/ml) in 10 mM sodium acetate (pH 5.5) was injected onto the activated sensor chip until the desired immobilization level (˜10000 RU) was achieved (3 min). Residual activated groups on the sensor chip were blocked by injection of ethanolamine (1 M, pH 8.5). Non-covalently bound conjugate was removed by repeated (5×) washing with 2.5 M NaCl, 50 mM NaOH. A second flow cell on the same sensor chip was treated identically, except with no Fc-c-Met immobilization, to serve as a control surface for refractive index changes and non-specific binding interactions with the sensor chip.

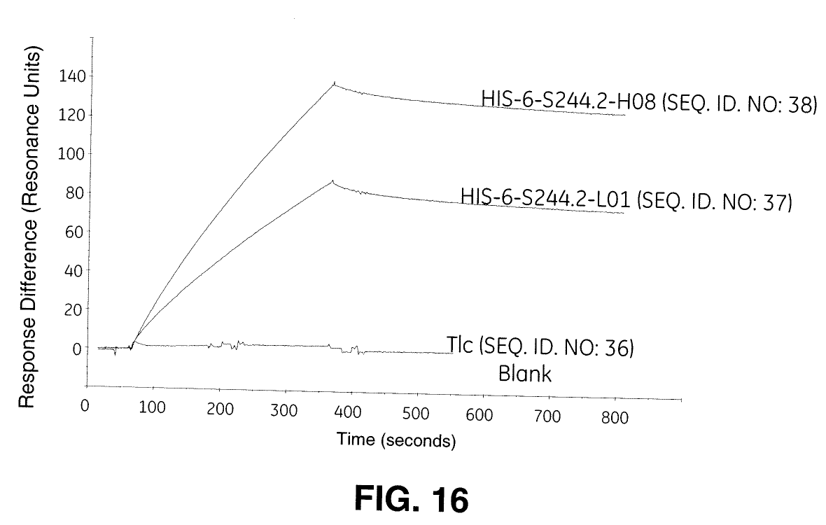

Before the kinetic study, binding of the target analyte was tested on both surfaces and a surface stability experiment was performed to ensure adequate removal of the bound analyte and regeneration of the sensor chip following treatment with 2.5 M NaCl, 50 mM NaOH. SPR sensorgrams were analyzed using the BIAevaluation software (GE Healthcare, Piscataway, N.J.). For the kinetics experiments, the binder flowed over the surface at 10 μL/min with an injection time of 5 min and dissociation time of 10 min. The surface was regenerated using 50 mM NaOH, 2.5 M NaCl buffer with injection time of 90 sec and stabilization time of 2 min. SPR measurements were collected at eight analyte concentrations (0-100 nM protein) and the resulting sensorgrams were fitted using a global separate fitting. Relative response for samples at a concentration of 100 nM is shown in FIG. 16.

### Example 23

### Radiolabeling via His6-Tag of Muteins hTLc (SEQ. ID. NO:36), S244.2-L01 (SEQ. ID. NO:37), and S244.2-H08 (SEQ. ID. NO:38)

Labeling of muteins with the fac-[^{99m}Tc(CO)₃]⁺ core was accomplished using modifications to a previously published procedure (as described in Waibel et al., Nat. Biotechnol. 1999, 17, 897.). Briefly, Na[^{99m}TcO₄] (Cardinal Health, Albany, N.Y.) in saline (4 mCi, 2 mL) was added to an Isolink® boranocarbonate kit (Mallinckrodt, see reference; Alberto, et al., J. Am. Chem. Soc. 2001, 123, 3135.). The resulting solution was heated to 95° C. for 15-20 minutes, to give fac-[^{99m}Tc(CO)₃(H2O)₃]⁺. A portion (2 mCi, 1 mL) of the solution was removed and neutralized to pH 7 with 122 mL of 1 N HCl and 10 mL of 0.1 M NaOH. A 300-μL aliquot was removed and added to a solution containing 90 mg of 6-His mutein. The resulting solution was heated in a water bath at 35° C. for 40 minutes. Radiochemical yields ranged from 67-73% (determined by ITLC-SG, Biodex, 0.9% NaCl). The crude reaction products were chromatographed on a NAP-5 column (GE Healthcare, 10 mM PBS) to give products of >99% radiochemical purity. Typical specific activities obtained were 3-4 mCi/mg. The resulting solution was then diluted with 10 mM PBS to give a final concentration of 0.01 mg/mL for subsequent biodistribution studies.

### Example 24

### In Vitro Binding of Lipocalin Muteins to c-Met Expressing Cells of His6-Tagged Proteins HIS-6-S244.2-H08 (SEQ. ID. NO:38) and HIS-6-S244.2-L01 (SEQ. ID. NO:37)

Two his-tagged lipocalin muteins, HIS-6-S244.2-H08 (SEQ. ID. NO:38) and HIS-6-S244.2-L01 (SEQ. ID. NO:37), were assayed for their affinity to intact cellular surfaces expressing c-Met cells HT29 cell (a human colorectal adenocarcinoma that expresses c-Met). C6 cells (a c-Met-negative rat glioma; ATCC, Manassas, Va.) were used as a negative control cell line. The proteins were labeled with ^{99m}Tc as described in Example 23 and measured for affinity to c-Met-expressing cells using an in vitro radiolabeled cell-binding assay as follows: HT29 and C6 cells were incubated in 24-well microtiter plates at 37° C., 5% CO₂, for 2-3 days at a concentration of 20,000 cells/well. Media was aspirated from wells and replaced with PBS. Each well of cells was then exposed to 15 nCi of ^{99m}Tc-labeled muteins for 60 min. Cells were subsequently washed with ice-cold PBS twice. Cells were then solubilized with 1 M NaOH and samples were collected and counted on a Perkin Elmer 1480 Gamma Counter. Percent bound was determined from the activity counted from the cells after washing and dividing by the total activity collected from the media and each of two washes.

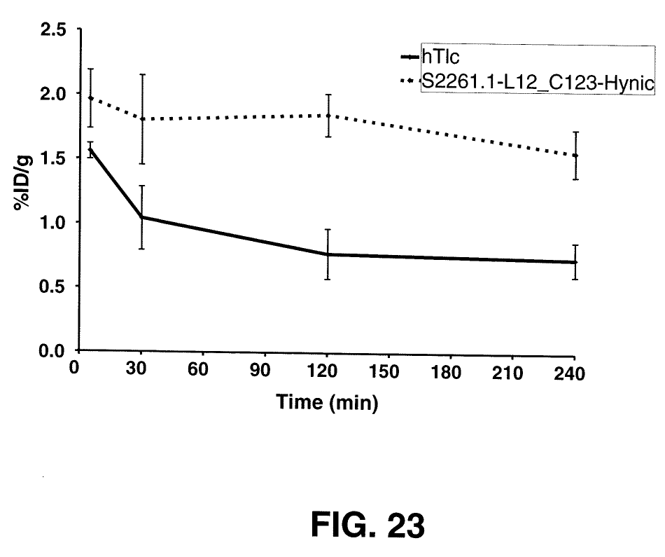

FIG. 23 shows that the lipocalin muteins HIS-6-S244.2-H08 (SEQ. ID. NO:38) and HIS-6-S244.2-L01 (SEQ. ID. NO:37) both demonstrated binding and an affinity to c-Met-expressing cells (HT29) as compared with the non-c-Met expressing cells (C6) with a fold increase of 17 and 18, respectively. This demonstrates that after radiolabeling, the muteins retain affinity to c-Met.

### Example 25

### In Vivo Targeted Studies of His6-Tagged Muteins HIS-6-S244.2-H08 and HIS-6-S244.2-L01 to c-Met-Expressing Lesions

Biodistribution studies were carried out in CD-1 nude mice (Charles River Labs, Hopkinton, Mass.) with an age range between 6 and 12 weeks. Mice were housed for at least 48 hours before biodistribution studies were carried out. Mice were inoculated with 3-5×10⁶ HT29 cells (ATCC, Manassas, Va.) suspended in Matrigel (BD Biosciences, San Jose, Calif.) and PBS. Tumor formation occurred in 3-4 weeks. Animals that had a tumor size from ˜100-200 mm3 were used for these studies.

Mice were given tail-vein injections of ˜1 μg of ^{99m}Tc-labeled mutein (˜3-5 mCi/1 mg) HIS-6-S244.2-H08 (SEQ. ID. NO:38) and HIS-6-S244.2-L01 (SEQ. ID. NO:37). Mice were placed in filter-paper lined cages until euthanasia. Three mice were euthanized at 5-, 30-, 120-, and 240-minute time points. Tissues of interest were dissected and counted on a Perkin Elmer 1480 Gamma Counter. Data were collected for blood, tumor, kidney, liver, spleen, and injection site (tail). Urine from cages was pooled with the bladder and also counted. The remaining tissues were counted and the sum of all tissues plus urine for each animal was summed to provide the total injected dose. The percent-injected dose for each organ was determined based on total injected dose, and organs were weighed for determination of the percent-injected dose per gram (% ID/g). Data is reported as mean value for all three mice in the time point with error bars representing the standard deviation of the group.

Three muteins, hTLc (SEQ ID NO:36 with a 6-his tag appended), HIS-6-S244.2-H08 (SEQ. ID. NO:38) and HIS-6-S244.2-L01 (SEQ. ID. NO:37) were labeled with ^{99m}Tc as described in Example 23 via 6-His tag and injected into HT29 tumor-bearing CD-1 nude mice to determine in vivo biodistribution and targeting to c-Met-expressing lesions.

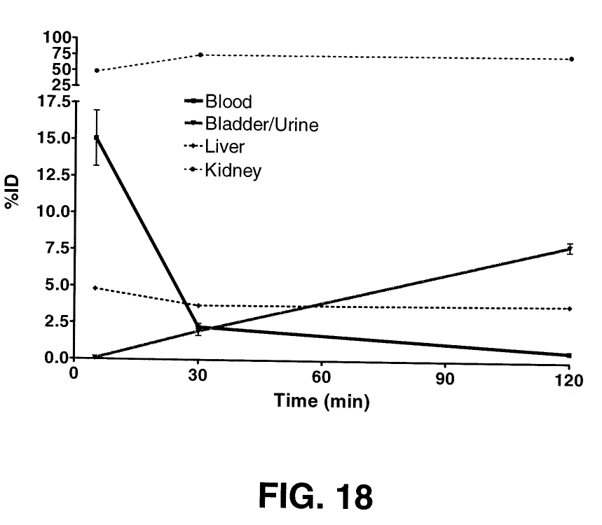

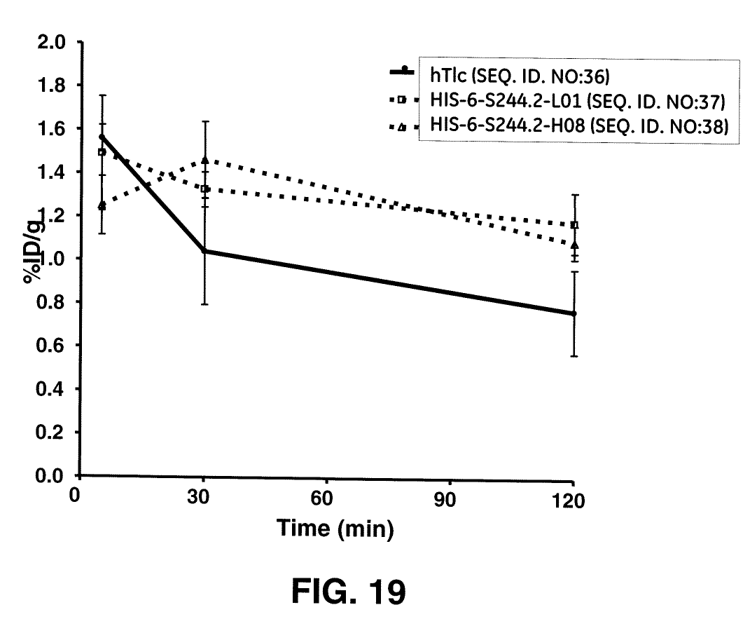

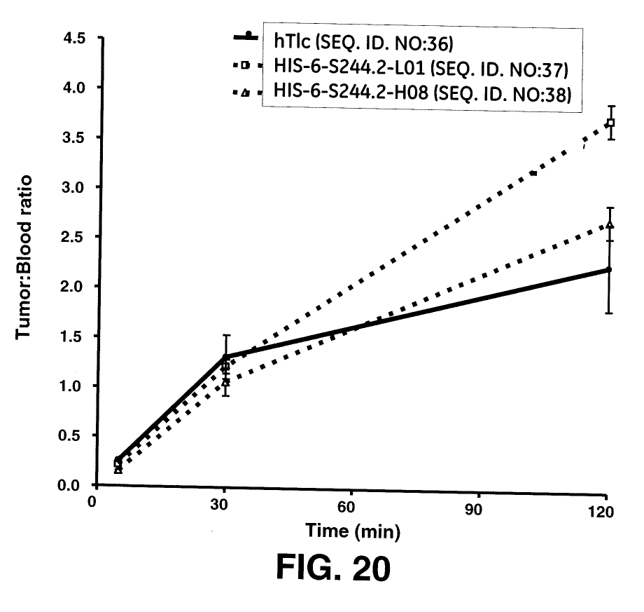

(SEQ ID NO:36 with a 6-his tag appended) is a negative control lipocalin mutein with no affinity for c-Met while both HIS-6-S244.2-H08 (SEQ. ID. NO:38) and HIS-6-S244.2-L01 (SEQ. ID. NO:40) display nanomolar affinity for c-Met FIG. 18 shows the clearance profile of HIS-6-S244.2-L01 (SEQ. ID. NO:37), typical of all 3 proteins, where the protein clears rapidly from the blood with a half-life of less than 10 minutes with a majority of the clearance through the kidneys. The lipocalin muteins also demonstrate moderate liver uptake in the range of 2.5 to 7.5% ID/g organ. The muteins HIS-6-S244.2-H08 (SEQ. ID. NO:38) and HIS-6-S244.2-L01 (SEQ. ID. NO:37) also target the c-Met expressing tumor rapidly more so than, hTLc (SEQ ID NO:36 with a 6-his tag appended) with uptake seen at 5 minutes post injection that is maintained through the end of the study. The percent-injected dose/g of tumor tissue are 0.77% for, hTLc (SEQ ID NO:36 with a 6-his tag appended, 1.09% for HIS-6-S244.2-H08 (SEQ. ID. NO:38), 1.18% for HIS_{—}6-S244.2-L01 (SEQ. ID. NO:37) at 2 hours post injection (FIG. 19). The tumor:blood ratio of the lipocalin muteins are shown in FIG. 20, hTLc (SEQ ID NO:36 with a 6-his tag appended), HIS_{—}6-S244.2-H08 (SEQ. ID. NO:38) and HIS_{—}6-S244.2-L01 (SEQ. ID. NO:37) are 2.261.63, 2.72, and 3.74, respectively. To demonstrate specificity of the mutein, ^{99m}Tc-radiolabeled HIS-6-S244.2-L01 (SEQ. ID. NO:37) at a concentration of ˜5 mCi/mg was injected with 100 mg of unlabeled HIS-6-S244.2-L01 (SEQ. ID. NO:37) to block the binding sites of c-Met on the tumor. FIG. 6 shows that the blocking with 100× cold mutein HIS-6-S244.2-L01 (SEQ. ID. NO:37) co-injected with the ^{99m}Tc-radiolabeled HIS-6-S244.2-L01 (SEQ. ID. NO:37) produced a 25% decrease in tumor uptake as compared against the radiolabeled HIS-6-S244.2-L01 (SEQ. ID. NO:37) only. Furthermore, none of the other organs demonstrated any significant decrease in non-specific uptake. This study shows that the targeted lipocalin muteins, HIS-6-S244.2-H08 (SEQ. ID. NO:38) and HIS-6-S244.2-L01 (SEQ. ID. NO:37), target c-Met-expressing lesions specifically in vivo.

### Example 26

### Radiolabeling via the His6-Tag of Muteins HIS-6-S2261.1-L12, HIS-6-S2261.1-L17, and HIS-6-S2261.1-J01

Labeling of HIS-6-S2261.1-L12 (SEQ. ID. NO:39), HIS-6-S2261.1-L17 (SEQ. ID. NO:41), and HIS-6-S2261.1-J01 (SEQ. ID. NO:40) with the fac-[^{99m}Tc(CO)₃]⁺ core was accomplished using modifications to the procedure described in Waibel, et al. (Nat. Biotechnol. 1999, 17, 897.). Briefly, Na[^{99m}TcO₄] in saline (4 mCi, 2 mL, Cardinal Health, Albany, N.Y.) was added to an Isolink® boranocarbonate kit (Mallinckrodt, see reference; Alberto, et al., J. Am. Chem. Soc. 2001, 123, 3135.). The resulting solution was heated to 95° C. for 15-20 minutes, to give fac-[^{99m}Tc(CO)₃(H₂O)₃]⁺. The entire solution was neutralized to pH 7.1 to 7.4 with 183 mL of 1 N HCl. A 300-pL aliquot was removed and added to a solution containing 50 mg of HIS-6-S2261.1-L12 (SEQ. ID. NO:39) in 25 mL distilled-deionized water. The resulting solution was heated in a water bath at 37° C. for 40 minutes. Typical radiochemical yields ranged from 51-83% (determined by ITLC-SG, Biodex, 0.9% NaCl). The crude reaction products were chromatographed on a NAP-5 column (GE Healthcare, 10 mM PBS) to give products of high radiochemical purity (>99%). An aliquot was removed for RP-HPLC and SEC-HPLC. Both analyses were performed with the same HPLC sample. Typical specific activities obtained were 10 mCi/mg. The resulting solution was then diluted with 10 mM PBS to give a final concentration of 0.01 mg/mL for subsequent biodistribution studies.

The HPLC conditions used for this experiment were: C4 RP-HPLC method 1 (His6/Hynic): Solvent A: 95/5H2O/CH3CN (with 0.05% TFA), Solvent B: 95/5 CH3CN/ddH2O with 0.05% TFA. Gradient elution: 0 min. 0% B; 4 min. 20% B; 16 min. 60% B; 20 min. 100% B; 25 min. 100% B; 26 min. 0% B; and 31 min. 0% B.

Analysis performed on an HP Agilent 1100 with a G1311A QuatPump, G1313A autoinjector with 100 mL syringe and 2.0 mL seat capillary, Grace Vydac protein C4 column (S/N E050929-2-1, 4.6 mm×150 mm), G1316A column heater, G1315A DAD and Ramon Star GABI gamma-detector.

SEC HPLC: Solvent: 1′ (10 mM) PBS (Gibco, Invitrogen, pH 7.4 containing CaCl₂ and MgCl₂). Isocratic elution for 30 min. Analysis performed on a: Perkin Elmer SEC-4 Solvent Environmental control, Series 410 LC pump, ISS 200 Advanced LC sample processor, and Series 200 Diode Array Detector. A Raytest GABI with Socket 8103 0111 pinhole (0.7 mm inner diameter with 250 mL volume) flow cell gamma detector was interfaced through a Perkin Elmer NCI-900 Network Chromatography Interface. The column used was a Superdex 75 10/300 GL High Performance SEC column (GE Healthcare, Piscataway, N.J., code: 17-5174-01, ID No. 0639059).

### Example 27

### In Vivo Targeting of His6-Tagged Muteins HIS-6-S2261.1-L12, HIS-6-S2261.1-L17, and HIS-6-S2261.1-J01 to c-Met-Expressing Lesions

Biodistribution studies were carried out in CD-1 nude mice (Charles River Labs, Hopkinton, Mass.) with an age range between 6 and 12 weeks. Mice were housed for at least 48 hours before biodistribution studies were carried out. Mice were inoculated with 3-5×10⁶ HT29 cells (ATCC, Manassas, Va.) suspended in Matrigel (BD Biosciences) and PBS. Tumor formation occurred in 3-4 weeks. Animals that had a tumor ˜100-200 mm³ used for these studies.

Mice were given tail-vein injections of ˜1 mg of ^{99m}Tc-labeled mutein (˜3-5 mCi/1 mg). Mice were placed in filter-paper lined cages until euthanasia. Three mice were euthanized at 5-, 30-, 120-, and 240-minute time points when tissues of interest were dissected and counted on a Perkin Elmer 1480 Gamma Counter. Data were collected for blood, kidney, liver, spleen, and injection site (tail). Urine from cages was pooled with the bladder and also counted. The remaining tissues were counted and the sum of all tissues plus urine for each animal was summed to provide the total injected dose. The percent-injected dose for each organ was determined based on this total, and organs were weighed for determination of the percent-injected dose per gram, (% ID/g). Data is reported as mean value for all three mice in the time point with error bars representing the standard deviation of the group.

Three muteins HIS-6-S2261.1-L12 (SEQ. ID. NO:39), S2261.1-L17 (SEQ. ID. NO:41), and HIS-6-S2261.1-J01 (SEQ. ID. NO:40) were injected into HT29 tumor-bearing CD-1 nude mice for determining in vivo biodistribution and targeting to c-Met expressing lesions. The proteins were labeled with ^{99m}Tc as described in Example 26.

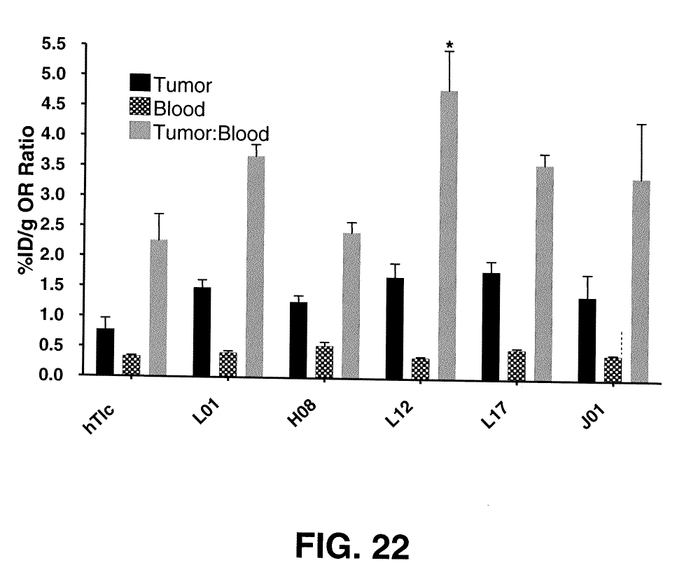

FIG. 22 shows that all three muteins target c-Met at 2 h post injection with tumor uptake significantly higher than the negative control protein, hTLc (SEQ ID NO:36). Furthermore, the tumor to blood ratio for HIS-6-S2261.1-L12 (SEQ. ID. NO:32) was statistically significant as compared to all of the other lipocalin muteins shown. These proteins also clear very rapidly from the blood with a half-life of less than 10 minutes and a majority of the clearance through the kidneys as seen previously. This study shows that the targeted lipocalin muteins target in vivo c-Met-expressing lesions.

### Example 28

### Bioconjugation of Mal-HYNIC with muteinS2261.1-L12_C123

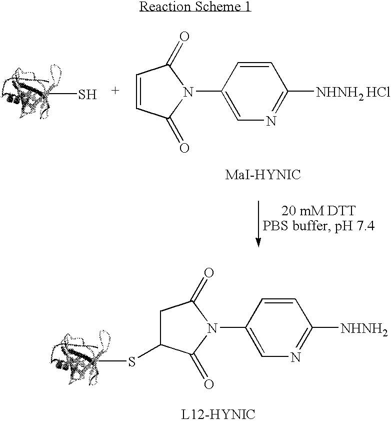

The general reaction scheme is shown in above in Reaction Scheme 1.

To a solution of S2261.1-L12_C123 (SEQ. ID. NO:35) mutein at a concentration of 0.8 mg/mL in PBS buffer (375 mL, 300 μg) was added 30 mL of a 1.35 M DTT solution in degassed PBS buffer to yield a final protein concentration of 100 mM. The mixture was vortexed at room temperature for 2.5 h. The DTT was removed by elution of the mixture through a Zeba Desalting Spin Column (Pierce Biotechnology) pre-equilibrated with degassed PBS buffer. To the eluant, 30.7 mL of an 8-mg/mL solution of Mal-HYNIC (N-(2-(2,5-dioxo-2,5-dihydro-1H-pyrrol-1-yl)ethyl)-6-hydrazinylnicotinamide hydrochloride) in DMSO was added to yield 15 equivalents of the Mal-HYNIC per mutein. The reaction mixture was vortexed at room temperature for 2 h. The conjugate was purified by elution through a Zeba Desalting Spin Column, removing excess of unreacted Mal-HYNIC. The conjugate was further purified with Size Exclusion Column (GEHC Superdex 75, 10/300 GL). The recovery yield was 47%. The expected product was obtained as characterized by MALDI-MS [M+Na]+ with a molecular weight of 17915 Da.

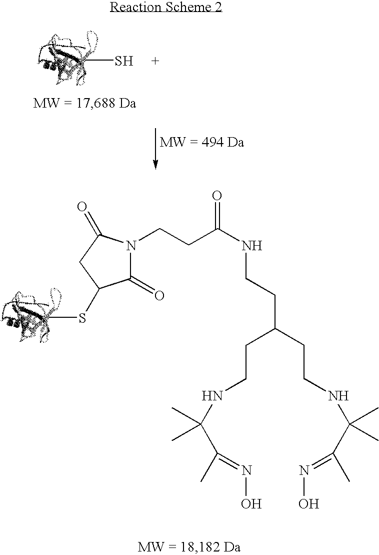

### Example 29

### Bioconjugation of Mal-cPn with S2261.1-L12_C123

The S2261.1-L12_C123 (SEQ. ID. NO:35) mutein (100 mg) was dissolved in 90 μL of degassed PBS buffer (pH 7.4). To the mutein solution, 10 mL of a 200 mM DTT solution in degassed PBS buffer were added to yield a final concentration of 20 mM. The mixture was vortexed at room temperature for 2 h. The DTT was removed by elution of the reduced L12 mutein mixture through a Zeba Desalting Spin Column (Pierce Biotechnology) preequilibrated with degassed PBS buffer. To the eluant, 2 mL of a 0.06 M solution of Mal-cPn linker in DMSO were added to yield 20 equivalents of the chelator per mutein. The reaction mixture was vortexed at room temperature for 2 h. The conjugate was purified by RP-HPLC with a 25% recovery yield. The expected product was obtained as characterized by MALDI-MS [M+Na]+demonstrating a molecular weight of 18182.19 Da. ESI-MS also confirmed the desired product corresponding to a molecular weight of 18187.8 Da.

### Example 30

### ^{99m}Tc Radiolabeling of HYNIC-S2261.1-L12_C123

Stannous chloride (100 mL of a 250 ug/mL⁻¹ in 0.1 N HCl) and tricine (200 mL of a 90 mg/mL⁻¹ in 10 mM PBS) were combined into a 25 mL centrifuge tube. The pH was adjusted to 6.8-7.0 with 0.1 N NaOH. To this mixture was added Na^{99m}TcO₄ (0.8 mCi, 29.6 MBq) in 0.080 mL of saline (0.15 M NaCl) obtained from Cardinal Health (Albany, N.Y.). The reaction was allowed to proceed for 20 min. at ambient temperature. ITLC (ITLC-SG, Biodex, 0.9% NaCl) analysis was performed to determine the extent of colloid formation (0.5-5%). 50 mg of S2261.1-L12_C123 (SEQ. ID. NO:35)-mal-Hynic was obtained as a lyophilized solid, and to this was added 350 mL of the ^{99m}Tc-tricine mixture. The reaction was maintained at ambient temperature for approximately 20 min. The crude radiochemical yield was determined by ITLC analysis (64-91%) followed immediately by size exclusion purification (NAP5, GEHC, charged with 10 mM PBS) of the reaction mixture. The reaction mixture (˜0.35 mL) was transferred, followed by a 0.1 mL of 10 mM PBS wash of the reaction vessel. The solution was allowed to enter the gel bed and the purified product isolated by eluting from the column with 0.8 mL of 10 mM PBS. The final radiochemical purity was analyzed by ITLC (95-96%), C4-RP HPLC and SEC HPLC. Typical specific activity was between 5 mCi/mg⁻¹ and 10 mCi/mg⁻¹.

C4 RP-HPLC method 1 (His6/Hynic): Solvent A: 95/5H2O/CH3CN (with 0.05% TFA), Solvent B: 95/5 CH3CN/ddH2O with 0.05% TFA. Gradient elution: 0 min. 0% B; 4 min. 20% B; 16 min. 60% B; 20 min. 100% B; 25 min. 100% B; 26 min. 0% B; and 31 min. 0% B.

Analysis performed on an HP Agilent 1100 with a G1311A QuatPump, G1313A autoinjector with 100 mL syringe and 2.0 mL seat capillary, Grace Vydac protein C4 column (S/N E050929-2-1, 4.6 mm×150 mm), G1316A column heater, G1315A DAD and Ramon Star-GABI gamma-detector.

C4 RP-HPLC method 2 (Hynic): Solvent A: 95/5H2O/CH3CN (with 0.05% TFA), Solvent B: 95/5 CH3CN/ddH2O with 0.05% TFA. Gradient elution: 0 min. 10% B; 30 min. 42% B; 35 min. 70% B; 36 min. 90% B; 40 min. 90% B; 45 min. 10% B; and 55 min. 10% B. Analysis was performed on an HP Agilent 1100 with a G1311A QuatPump, G1313A autoinjector with 100 mL syringe and 2.0 mL seat capillary, Grace Vydac protein C4 column (S/N E050929-2-1, 4.6 mm×150 mm), G1316A column heater, G1315A DAD, and Ramon Star GABI gamma-detector.

SEC HPLC: Solvent: 1 (10 mM) PBS (Gibco, Invitrogen, pH 7.4 containing CaCl2 and MgCl2). Isocratic elution for 30 min. Analysis performed on a: Perkin Elmer SEC-4 Solvent Environmental control, Series 410 LC pump, ISS 200 Advanced LC sample processor, and Series 200 Diode Array Detector. A Raytest GABI with Socket 8103 0111 pinhole (0.7 mm inner diameter with 250 mL volume) flow cell gamma detector was interfaced through a Perkin Elmer NCl-900 Network Chromatography Interface. The column used was a Superdex 75 10/300 GL High Performance SEC column (Code: 17-5174-01, ID No. 0639059).

### Example 31

### In Vivo Targeting of S2261.1-L12_C123 (SEQ. ID. NO:35) HYNIC-^{99m}Tc-Labeled Lipocalin Mutein in c-Met-Expressing Lesions

Biodistribution studies were carried out in CD-1 nude mice (Charles River Labs, Hopkinton, Mass.) with an age range between 6 and 12 weeks. Mice were housed for at least 48 hours before biodistribution studies were carried out. Mice were inoculated with 3-5×10⁶ HT29 cells (ATCC, Manassas, Va.) suspended in Matrigel (BD Biosciences, San Jose, Calif.) and PBS. Tumor formation occurred in 3-4 weeks. Animals that had a tumor ˜100-200 mm³ were used for these studies.

Mice were given tail-vein injections of ˜1 mg of ^{99m}Tc-labeled mutein S2261.1-L12_C123 (SEQ. ID. NO:35) (˜3-5 mCi/1 mg). Mice were placed in filter-paper lined cages until euthanasia. Three mice were euthanized at 5-, 30-, 120-, and 240-minute time points. Tissues of interest were dissected and counted on a Perkin Elmer 1480 Gamma Counter. Data were collected for blood, tumor, kidney, liver, spleen, and injection site (tail). Urine from cages was pooled with the bladder and also counted. The remaining tissues were counted and the sum of all tissues plus urine for each animal was summed to provide the total-injected dose. The percent-injected dose for each organ was determined based on this total, and organs were weighed for determination of the percent-injected dose per gram, (% ID/g). Data is reported as mean value for all three mice in the time point with error bars representing the standard deviation of the group.

S2261.1-L12_C123 (SEQ. ID. NO:35) labeled with ^{99m}Tc via HYNIC was injected into HT29 tumor-bearing CD-1 nude mice for determining in vivo biodistribution and targeting to c-Met expressing lesions. The protein was labeled with ^{99m}Tc as described in Example 30.

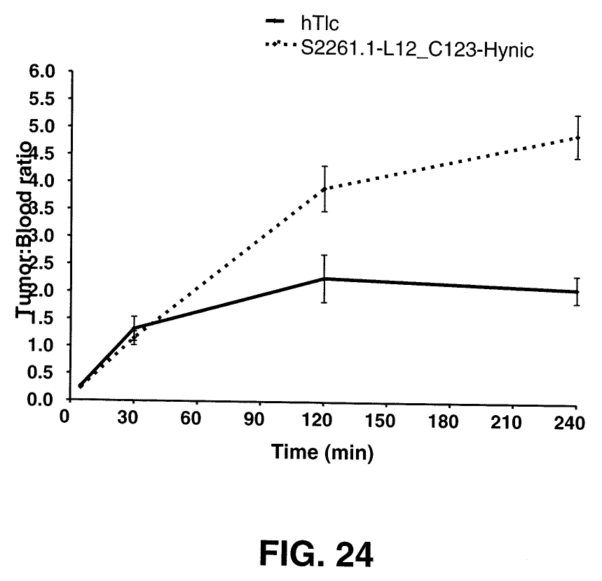

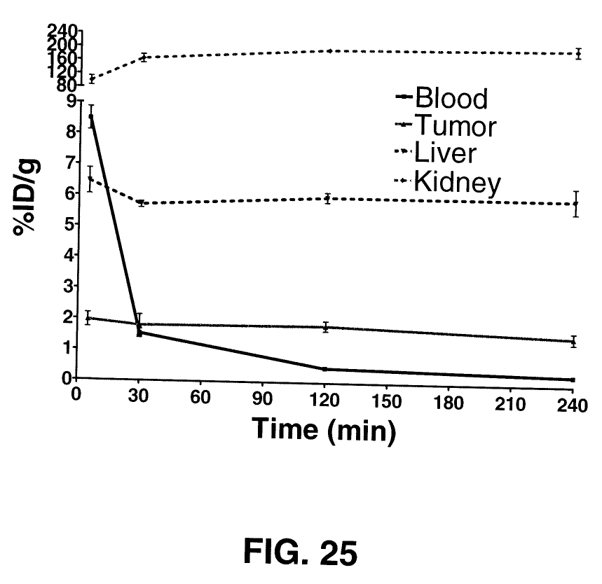

FIG. 23 shows that S2261.1-L12_C123 (SEQ. ID. NO:35) labeled with ^{99m}Tc via HYNIC targets the c-Met-expressing tumor rapidly with uptake seen at 5 minutes post injection. The uptake values are approximately maintained through the end of the study at 4 hours post injection with a percent-injected dose/g of 0.73% for hTLc (SEQ ID NO:36 with a 6-his appended) and 1.56% for S2261.1-L12_C123 (SEQ. ID. NO:35) labeled with ^{99m}Tc via HYNIC. The tumor:blood ratio of the lipocalin muteins, hTLc (SEQ ID NO:36) and L12-HYNIC, were 2.09 and 4.90, respectively (FIG. 24). FIG. 25 shows that L12-HYNIC also clears rapidly from the blood with a half-life of less than 10 minutes with a majority of the clearance through the kidneys. This study shows that the targeted lipocalin mutein labeled with HYNIC targets c-Met-expressing lesions in vivo.

### Example 32

### ^{99m}Tc Labeling of S2261.1-L12_C123 (SEQ. ID. NO:35) via cPn

Synthesis of the radiolabeled mutein was performed using a one-step kit formulation (Chelakit A+, GEHC) containing a lyophilized mixture of stannous chloride as a reducing agent for technetium, methylene diphosphonic acid, and p-aminobenzoate as a free-radical scavenger and sodium bicarbonate/sodium carbonate (pH 9.2) as buffer. In rapid succession, 40 mL of a 0.625 mg/mL-1 solution of mutein in saline was added to the Chelakit A+, followed immediately by Na^{99m}TcO₄ (0.8 mCi, 29.6 MBq) in 0.080 mL of saline (0.15 M NaCl) obtained from Cardinal Health (Albany, N.Y.). The mixture was agitated once and allowed to sit at ambient temperature for 20 min. Upon completion, the crude radiochemical yield was determined by ITLC (69%, Dark-Green, Biodex) and C4-RP HPLC. The reaction volume was brought to 0.45 mL with 0.33 mL of saline and the final product was purified by size exclusion chromatography (NAP5, GEHC, charged with 10 mM PBS). The sample was loaded and allowed to enter the gel bed, and the final purified product isolated by eluting with 0.8 mL of 10 mL PBS. Final activity was assayed in a standard dose calibrator (CRC-15R, Capintec, Ramsey, N.J.). Radiochemical yield and purity were determined by ITLC (84%), C4 RP-HPLC and SEC-HPLC analysis.

The HPLC conditions used for this experiment were: C4 RP-HPLC method 3(cPn216): Solvent A: H₂O with 0.06% NH3, Solvent B: CH₃CN. Gradient elution: 0 min. 0% B; 4 min. 20% B; 16 min. 60% B; 20 min. 100% B; 25 min. 100% B; 26 min. 0% B; 31 min. 0% B. Analysis performed on an HP Agilent 1100 with a G1311A QuatPump, G1313A autoinjector with 100 mL syringe and 2.0 mL seat capillary, Grace Vydac protein C4 column (S/N E050929-2-1, 4.6 mm×150 mm), G1316A column heater, G1315A DAD, and Ramon Star GABI gamma-detector.

SEC HPLC: Solvent: 1′ (10 mM) PBS (Gibco, Invitrogen, pH 7.4 containing CaCl2 and MgCl2). Isocratic elution for 30 min. Analysis performed on a: Perkin Elmer SEC-4 Solvent Environmental control, Series 410 LC pump, ISS 200 Advanced LC sample processor, and Series 200 Diode Array Detector. A Raytest GABI with Socket 8103 0111 pinhole (0.7 mm inner diameter with 250 mL volume) flow cell gamma detector was interfaced through a Perkin Elmer NCl-900 Network Chromatography Interface. The column used was a Superdex 75 10/300 GL High Performance SEC column (code: 17-5174-01, ID No. 0639059).

### Example 33

### Bioconjugation of Aminoxy Linker to Mutein S2261.1-L12-C123 (SEQ. ID. NO:35)

The general reaction scheme shown below in Reaction Scheme 3.

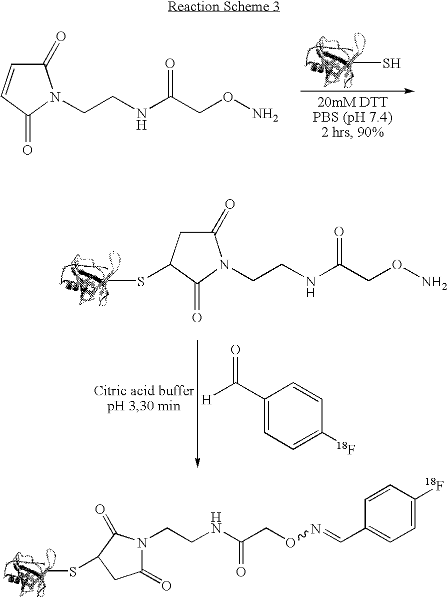

The S2261.1-L12-C123 (SEQ. ID. NO:35) mutein (100 mg) is dissolved with 90 mL of degassed PBS buffer (pH 7.4). To the mutein solution, 10 mL of a 200 mM DTT solution in degassed PBS buffer is added to yield a final concentration of 20 mM. The mixture is vortexed at room temperature for 2 h. The DTT is removed by elution of the mixture through a Zeba Desalting Spin Column (Pierce Biotechnology). To the eluant, 5 mL of a Mal-Aminoxy solution in DMSO is added so that it yields 20 equivalents of the linker per mutein. The reaction mixture is vortexed at room temperature for 2 h. The conjugate is purified by RP-HPLC and is ready for subsequent reaction with an 18F-containing aldehyde synthon as shown in the Reaction Scheme 2.

### Example 34

### Bioconjugation of Cyanine Dye with His6-Tag-Free Mutein L12 to Produce S2261.1-L12-C123 (SEQ. ID. NO:35)-Cy5.5

The S2261.1-L12-C123 (SEQ. ID. NO:35) (100 mg) was dissolved with 100 mL of degassed PBS buffer (pH 7.4). To the mutein solution, 10 mL of a 1.35 M DTT solution in degassed PBS buffer was added to yield a final concentration of 100 mM. The mixture was vortexed at room temperature for 2 h. The DTT was removed by elution of the reduced mutein mixture through a Zeba Desalting Spin Column (Pierce Biotechnology) preequilibrated with degassed PBS buffer. To the eluant, 12.2 mL of a 6.7-mg/mL solution of Mal-Cye5.5 in DMSO was added to yield 15 equivalents of the dye per mutein. The reaction mixture was vortexed at room temperature for 2 h. The conjugate was purified by elution through a Zeba Desalting Spin Column preequilibrated with MilliQ water, removing excess of unbound dye. The conjugate recovery yield was 79%. The expected product was obtained as characterized by MALDI-MS [M+Na]+ demonstrating a molecular weight of 18467.00 Da.

### Example 35

### Bioconjugation of Cyanine Dye with hTLc (SEQ ID NO:36) to Produce TLPC-Cye5.5

A solution of hTLc (SEQ ID NO:36) at a concentration of 2.6 mg/mL in PBS buffer (38.5 mL, 100 mg) was diluted by the addition of 61.5 mL of a 0.2 M NaHCO3 yielding a pH between 8-9. A solution of NHS-Cye5.5 was prepared with DMSO at a 1.8 mM concentration and 10 μL of this solution were added to the hTLc (SEQ ID NO:36) solution to yield 3 equiv of dye per hTLc (SEQ ID NO:36) molecule. The mixture was vortexed at room temperature for 7 h. The excess of free dye was removed by elution of the conjugate trough a Zeba Desalting Spin Column. MilliQ water was used for both preequilibration of the column and elution of the conjugate. A mixture of non-labeled, 1 dye, 2 dyes and 3 dye-conjugates was obtained. The conjugate mixture recovery yield was 85%. The expected products were obtained as characterized by MALDI-MS [M+Na]+ demonstrating molecular weights of 17507.03, 18149.86, 18794.14, 19014.53 Da corresponding to the expected single, double and triple functionalization of the dye via the multiple amines on the hTLc (SEQ ID NO:36) protein.

### Example 36

### Biodistribution of S2261.1-L12-C123 (SEQ. ID. NO:35)—Cye5.5 and TLPC-Cye5.5 in c-Met-Expressing Lesions

In vivo targeting and binding of lipocalin muteins using various modalities including optical imaging, to c-Met expressing lesions are shown.

Biodistribution studies were carried out in CD-1 nude mice (Charles River Labs, Hopkinton, Mass.) with an age range between 6 and 12 weeks. Mice were housed for at least 48 hours before biodistribution studies were carried out. Mice were inoculated with 3−5×10⁶ HT29 cells (ATCC, Manassas, Va.) suspended in Matrigel (BD Biosciences) and PBS. Tumor formation occurred in 3-4 weeks when the size of the tumor was 100-200 mm³. These mice were then used for these studies.

Mice were given tail-vein injections of ˜1 μg of Cy5-labeled muteins, S2261.1-L12-C123 (SEQ. ID. NO:35) and hTLc (SEQ ID NO:36). Animals were imaged at each time point using a GE Optix scanner (GEHC/ART, Milwaukee, Wis.). Whole-body (from the neck to the base of the tail) imaging protocols used a 635 nm laser operating at 20 mW, 1.5 mm sampling and 0.5 seconds integration time. Animals were anesthetized using 2-3% isofluorane gas and kept warm by a 37° C.-heated platform while imaged. Imaging was performed with the animals in a prone position to ensure that the xenograft tumor was well within the field of view of the scanner. Total acquisition times were about 3 minutes. Regions of interest (ROls) were drawn on the tumor images. The average fluorescent intensity was calculated from each 12 pixel ROI.

A similar processing was applied to background ROIs selected from the region contralateral to the tumor. Tumor:background ratio was determined from the fluorescent intensity of the tumor divided by the intensity from the contralateral region. Data is reported as mean value for three mice with error bars representing the standard deviation of the group.

S2261.1-L12-C123 (SEQ. ID. NO:35) labeled with Cye5.5 was injected into HT29 tumor-bearing CD-1 nude mice to determine in vivo biodistribution and targeting to c-Met-expressing lesions. hTLc (SEQ ID NO:36) labeled with Cy5.5 was injected as a negative control.

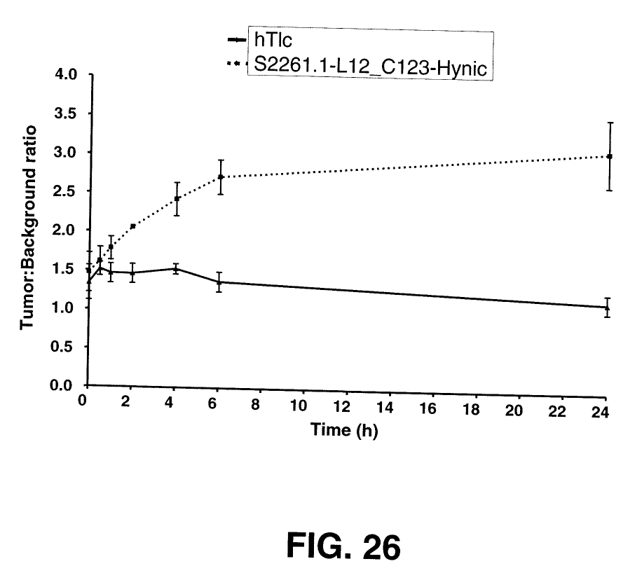

S2261.1-L12-C123 (SEQ. ID. NO:35) labeled with Cye5.5 lipocalin mutein targets the c-Met-expressing tumor rapidly with uptake seen at 5 minutes post injection that was maintained through the end of the study. This is in contrast to the negative control protein, hTLc (SEQ. ID. NO:36) labeled with Cy5.5, which does not significantly target c-Met as compared to background or non-specific uptake. The tumor:background ratio of the lipocalin muteins hTLc (SEQ. ID. NO:36) and S2261.1-L12-C123 (SEQ. ID. NO:35) labeled with Cy5.5, were 1.15 and 3.10, respectively at 24 h post injection (FIG. 26). This study shows that the targeted lipocalin mutein, S2261.1-L12 (SEQ. ID. NO:32) labeled with Cye5.5 targets c-Met-expressing lesions in vivo.

The invention may be embodied in other specific forms without departing from the spirit or essential characteristics thereof. The foregoing embodiments are therefore to be considered in all respects as illustrative rather than limiting on the invention described herein. The scope is thus indicated by the appended claims rather than by the foregoing description, and all changes that come within the meaning and range of equivalency of the claims are therefore intended to be embraced therein.

What is claimed is:

1. An in vivo imaging agent comprising a mutein of hTLc having detectable binding affinity for c-Met or a domain thereof, wherein the mutein comprises amino acid replacements for at least one sequence position corresponding to sequence positions 26-34, 56-58, 80, 83, 104-106, and 108 of hTLc of SEQ. ID. NO:36, the mutein is coupled to a signal generator, and the mutein coupled to a signal generator is disposed in a pharmaceutically acceptable carrier.

2. The in vivo imaging agent of claim 1, wherein the mutein comprises at least 2, 3, 4, 5, 6, 8, 10, 12, 14, 16 or 18 mutated amino acid residues at the sequence positions 26-34, 56-58, 80, 83, 104-106, or 108 of SEQ. ID. NO: 36.

3. The in vivo imaging agent of claim 2, wherein the mutein comprises mutated amino acid residues at sequence positions 26, 27, 28, 30, 31, 32, 33, 34, 56, 57, 58, 80, 83, 104, 105, 106, or 108 of SEQ. ID. NO: 36.

4. The in vivo imaging agent of claim 3, wherein the mutein additionally comprises at least one of the amino acid substitutions Cys 61→Ser, Cys 101→Ser, or Cys 153→Ser of SEQ. ID. NO: 36.

5. The in vivo imaging agent of claim 4, wherein the mutein comprises at least one additional amino acid substitution selected from Arg 111→Pro or Lys 114→Trp.

6. The in vivo imaging agent of claim 1, wherein the mutein comprises an amino acid sequence of SEQ. ID. NOss: 1, 4-9, 22-26-32-35 and 37-41.

7. The in vivo imaging agent of claim 1, wherein the signal generator comprises a radionuclide, a paramagnetic ion, a chemiluminescent agent, or a fluorophore.

8. The in vivo imaging agent of claim 7, wherein the radionuclide is selected from ¹¹C, ¹⁸F, ⁶⁷Ga, ⁶⁸Ga, ^{94m}Tc, ^{99m}Tc, ⁶⁴Cu, ⁶⁷Cu, ¹²³I, or ¹²⁴I.

9. The in vivo imaging agent of claim 8, wherein the radionuclide is ¹⁸F, ¹²³I, or ¹²⁴I that is attached to the mutein via an aminoxy group.

10. The in vivo imaging agent of claim 9, wherein the aminoxy group is bound to a thiol-containing amino acid residue present in the mutein.

11. The in vivo imaging agent of claim 8, wherein the radionuclide is ^{94m}Tc or ^{99m}Tc attached to the mutein via an imidazole-containing tag selected from tris-histidine or hexa-histidine (SEQ ID NO: 42).

12. The in vivo imaging agent of claim 8, wherein the radionuclide is ^{94m}Tc or ^{99m}Tc that is attached to the mutein via a linker selected from a bis amine-oxime or hydrazine nicotinic acid.

13. The in vivo imaging agent of claim 11, wherein the radionuclide is ^{94m}Tc or ^{99m}Tc that is attached to the mutein via a linker selected cPn216 or HYNIC.

14. The in vivo imaging agent of claim 13, wherein linker is bound to a thiol-containing amino acid residue present in the mutein.

15. The in vivo imaging agent of claim 8, wherein the radionuclide is selected from ⁶⁷Ga, ⁶⁸Ga, ⁶⁴Cu, or ⁶⁷Cu that is attached to the mutein via a chelator selected from NOTA, DOTA, or DTPA.

16. The in vivo imaging agent of claim 15, wherein the chelator is bound to a thiol-containing amino acid residue present in the mutein.

17. The in vivo imaging agent of claim 8, wherein the fluorescent agent is a cyanine dye or a quantum dot.

18. The in vivo imaging agent of claim 8, wherein the fluorescent agent is bound to a thiol-containing amino acid residue present in the mutein.

19. The in vivo imaging agent of claim 8, wherein the chemiluminescent agent is selected from green fluorescent protein, yellow fluorescent protein, or luciferin.

20. A method of detecting a cell proliferative disorder in a mammalian subject comprising:
(a) administering an in vivo imaging agent comprising a mutein of hTLc having detectable binding affinity for c-Met or a domain thereof, wherein the mutein comprises amino acid replacements for at least one sequence position corresponding to sequence positions 26-34, 56-58, 80, 83, 104-106, and 108 of hTLc of SEQ. ID. NO:36, the mutein is coupled to a signal generator, and the mutein coupled to a signal generator is disposed in a pharmaceutically acceptable carrier; and
(b) observing the signal produced by the in vivo agent.

21. The method of claim 20, wherein the cell proliferative disorder is selected from liver cancer, colon cancer, colorectal cancer, hepatocellular carcinoma, papillary renal carcinoma, head and neck squamous cell carcinoma (HNSC), lymph nodes metastases of head and neck, or squamous carcinoma.

22. The method of claim 20, wherein the radionuclide is selected from ¹¹C, ¹⁸F, ⁶⁸Ga, ¹²⁴I, ⁶⁴Cu, or ^{94m}Tc and the signal is observed using positron emission tomography.

23. The method of claim 20, wherein the radionuclide is selected from ^{99m}Tc, ⁶⁷Ga, ¹²³I, or ⁶⁷Cu and the signal is observed using single photon emission computed tomography.

24. The method of claim 20, wherein the paramagnetic ion is selected from ¹⁵⁷Gd, ⁵⁵Mn, ¹⁶²Dy, ⁵²Cr, or ⁵⁶Fe and the signal is observed using magnetic resonance.

25. The method of claim 20, wherein the signal generator is a fluorophore selected from a cyanine dye or a quantum dot and the signal is observed using optical imaging.

26. The method of claim 20, wherein the mutein comprises an amino acid sequence of SEQ. ID. NOs:1, 4-9, 22-26-32-35, or 37-41.

27. The method of claim 20, wherein the mutein is administered to the mammalian subject parenterally via intracutaneous, subcutaneous, intramuscular, intratracheal, intranasal, intravitreal, or intravenous injection or infusion.
